(19) United States
(12) Patent Application Publication
(10) Pub. No.: US 2025/0177231 A1
(43) Pub. Date: Jun. 5, 2025

(54) METHOD FOR DETERMINING GAIT STATE, AND DEVICE PERFORMING METHOD

(71) Applicant: SAMSUNG ELECTRONICS CO., LTD., Suwon-si (KR)

(72) Inventors: Yunjung KIM, Suwon-si (KR); Pyeonggook JUNG, Suwon-si (KR)

(21) Appl. No.: 19/051,933

(22) Filed: Feb. 12, 2025

Related U.S. Application Data
(63) Continuation of application No. PCT/KR2023/010098, filed on Jul. 14, 2023.

(30) Foreign Application Priority Data
Aug. 12, 2022 (KR) 10-2022-0101230
Oct. 12, 2022 (KR) 10-2022-0130253

(51) Int. Cl. A61H 3/00; A61H 1/02; B25J 9/00

(52) U.S. Cl. CPC A61H 3/00; A61H 1/0244; B25J 9/0006; A61H 2201/1215; A61H 2201/5069; A61H 2201/5084

(57) ABSTRACT

An electronic device may: determine a first interior angle on the basis of a first angle of a first joint and a first angle of a second joint of a wearable device, wherein the first angle of the first joint and the first angle of the second joint correspond to a first viewpoint; determine a second interior angle on the basis of a second angle of the first joint and a second angle of the second joint of the wearable device, wherein the second angle of the first joint and the second angle of the second joint correspond to a second viewpoint; determine the amount of change in a target interior angle between the first viewpoint and the second viewpoint on the basis of the first interior angle and the second interior angle; and determine a user's gait state on the basis of the amount of change in the target interior angle.

### CROSS-REFERENCE TO RELATED APPLICATIONS

This application is a continuation application of International Application No. PCT/KR2023/010098 designating the United States, filed on Jul. 14, 2023, in the Korean Intellectual Property Receiving Office and claiming priority to Korean Patent Application No. 10-2022-0101230, filed on Aug. 12, 2022, and Korean Patent Application No. 10-2022-0130253, filed on Oct. 12, 2022, the disclosures of which are all hereby incorporated by reference herein in their entireties.

### BACKGROUND

### Technical Field

Various example embodiments relate to a technology for determining a gait state.

### Description of Related Art

A change into aging societies has contributed to a growing number of people who experience inconvenience and pain from reduced muscular strength or joint problems due to aging or the like. Thus, there is a growing interest in walking assist devices that enable elderly users or patients with reduced muscular strength or joint problems to walk with less effort, and/or which allows users to exercise.

### SUMMARY

According to an example embodiment, an electronic device may include a communication module, comprising communication circuitry, configured to exchange data with an external device, and at least one processor, comprising processing circuitry, configured to control the electronic device, and the processor may be configured to perform an operation of determining a first internal angle based on a first angle of a first joint and a first angle of a second joint of a wearable device corresponding to a first time point, wherein the first joint corresponds to an angle of a hip joint of a first leg of a user wearing the wearable device, and the second joint corresponds to an angle of a hip joint of a second leg of the user, an operation of determining a second internal angle based on a second angle of the first joint and a second angle of the second joint of the wearable device corresponding to a second time point, an operation of determining a target internal angle change between the first time point and the second time point based on the first internal angle and the second internal angle, and an operation of determining a gait state of the user based on the target internal angle change.

According to an example embodiment, a method of determining a gait state, performed by an electronic device, may include an operation of determining a first internal angle based on a first angle of a first joint and a first angle of a second joint of a wearable device corresponding to a first time point, wherein the first joint corresponds to an angle of a hip joint of a first leg of a user wearing the wearable device, and the second joint corresponds to an angle of a hip joint of a second leg of the user, an operation of determining a second internal angle based on a second angle of the first joint and a second angle of the second joint of the wearable device corresponding to a second time point, an operation of determining a target internal angle change between the first time point and the second time point based on the first internal angle and the second internal angle, and an operation of determining a gait state of the user based on the target internal angle change.

According to an example embodiment, a wearable device may include at least one sensor configured to measure an angle of a thigh support frame, a motor driver circuit controlled by the processor, a motor electrically connected, directly or indirectly, to the motor driver circuit, and the thigh support frame configured to transmit a torque generated by the motor to at least a portion of a lower limb of the user, and the processor may be configured to perform an operation of determining a first internal angle based on a first angle of a first joint and a first angle of a second joint of the wearable device corresponding to a first time point, an operation of determining a second internal angle based on a second angle of the first joint and a second angle of the second joint corresponding to a second time point, an operation of determining a target internal angle change between the first time point and the second time point based on the first internal angle and the second internal angle, an operation of determining a gait state of the user based on the target internal angle change, and an operation of controlling an operation of the wearable device based on the gait state.

### BRIEF DESCRIPTION OF DRAWINGS

FIG. 1 is a diagram illustrating a configuration of a system for providing a user with a workout program according to an example embodiment.

FIG. 2 is a block diagram of an electronic device in a network environment according to an example embodiment.

FIGS. 3A, 3B, 3C, and 3D are diagrams illustrating a wearable device according to an example embodiment(s).

FIG. 4 is a diagram illustrating a wearable device communicating with an electronic device according to an example embodiment.

FIGS. 5 and 6 are diagrams illustrating a torque output method of a wearable device according to an example embodiment.

FIG. 7 is a diagram illustrating various gaits according to an example embodiment.

FIG. 8 is a flowchart of a method of determining a gait state according to an example embodiment.

FIG. 9A is a diagram illustrating sensing data of hip joints for a normal gait according to an example embodiment.

FIG. 9B is a diagram illustrating sensing data of hip joints for an abnormal gait according to an example embodiment.

FIG. 10 is a flowchart of a method of determining a gait state of a user based on a target internal angle change according to an example.

FIG. 11 is a flowchart of a method of determining a gait state of a user based on an internal angle change trajectory according to an example.

FIG. 12 is a diagram illustrating a predetermined stride event according to an example.

FIG. 13 is a flowchart of a method of determining a gait state of a user based on an internal angle change trajectory according to an example.

FIG. 14 is a diagram illustrating sensing data for determining a gait state of a user based on a peak time point according to an example.

FIG. 15 is a diagram illustrating sensing data for determining a gait state of a user based on a peak time point according to an example.

FIG. 16 is a diagram illustrating sensing data for determining a gait state of a user based on a peak time point according to an example.

FIG. 17 is a flowchart of a method of controlling an operation of a wearable device according to an example embodiment.

### DETAILED DESCRIPTION

Hereinafter, various example embodiments of the present disclosure will be described with reference to the accompanying drawings. However, this is not intended to limit the present disclosure to specific embodiments, and it should be understood that various modifications, equivalents, and/or alternatives of the embodiments of the present disclosure are included.

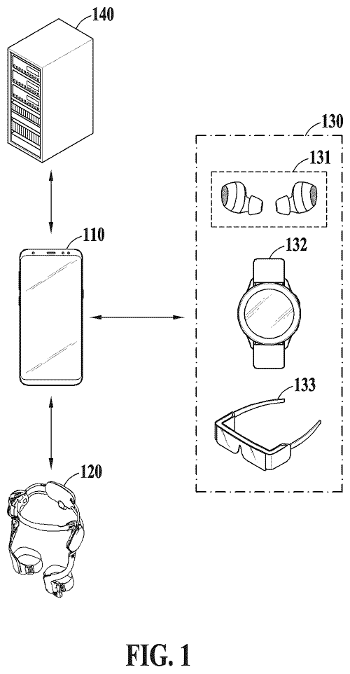

FIG. 1 is a diagram illustrating a configuration of a system for providing a user with a workout program according to an embodiment.

According to an embodiment, a system 100 for providing a user with a workout program may include an electronic device 110, a wearable device 120, an additional device 130, and a server 140.

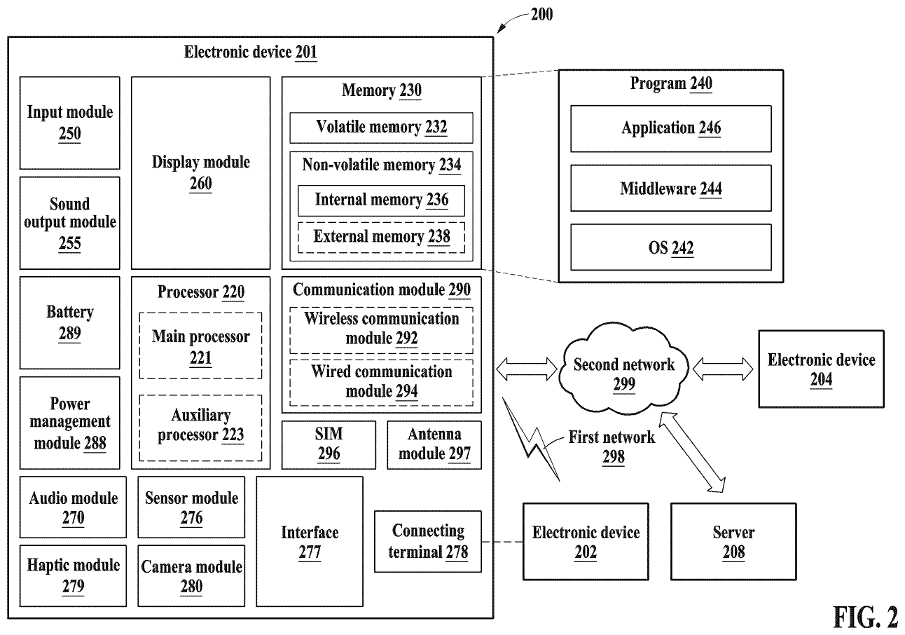

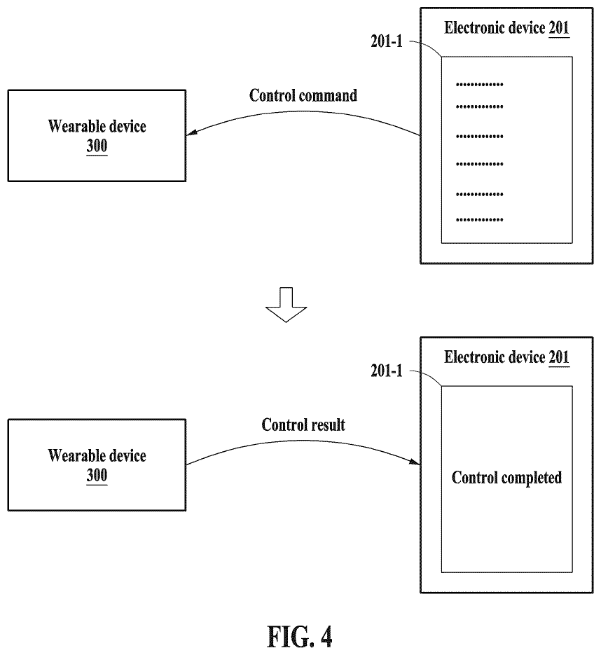

According to an embodiment, the electronic device 110 may be a user terminal that may be connected to the wearable device 120 using short-range wireless communication. For example, the electronic device 110 may transmit a control signal for controlling the wearable device 120 to the wearable device 120. The electronic device 110 will be described in detail below with reference to FIG. 2, and the transmission of a control signal will be described in detail below with reference to FIG. 4.

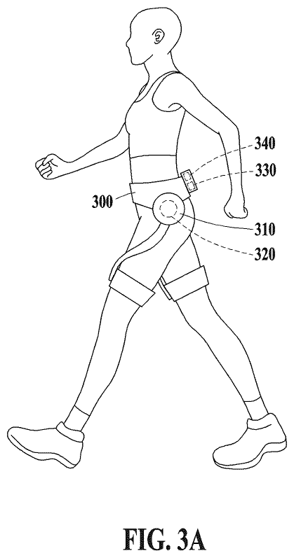

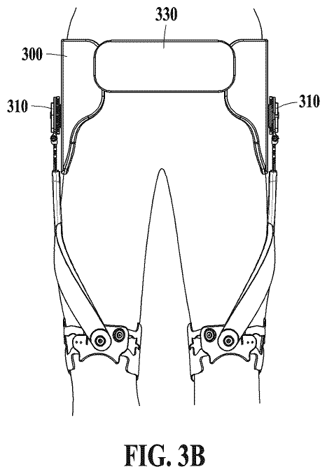

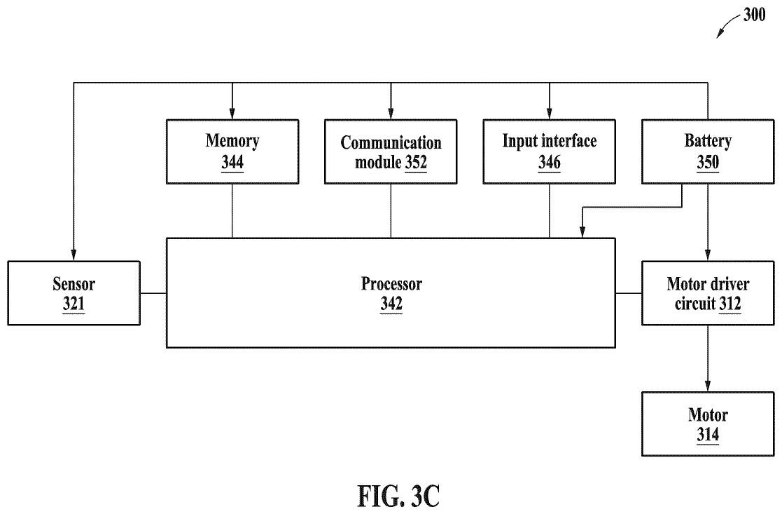

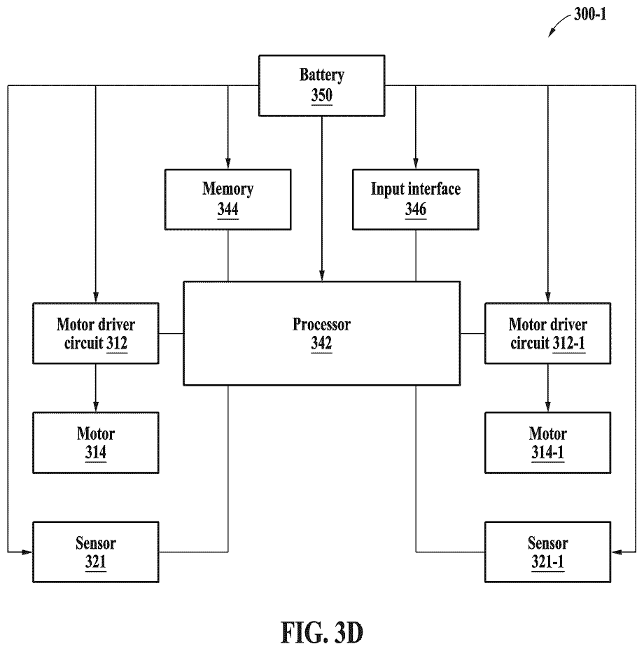

According to an embodiment, the wearable device 120 may provide a user wearing the wearable device 120 with an assistance force for assisting a gait or a workout or a resistance force for impeding a gait. The resistance force may be provided to the user to assist the user in doing a workout. The values of various control parameters used in the wearable device 120 may be controlled to control the assistance force or the resistance force output by the wearable device 120. The structure and driving method of the wearable device 120 will be described in detail below with reference to FIGS. 3A, 3B, 3C, 3D, 4, 5, and 6.

According to an embodiment, the electronic device 110 may be connected to the additional device 130 (e.g., wireless earphones 131, a smart watch 132, or smart glasses 133) using short-range wireless communication. For example, the electronic device 110 may output information indicating the state of the electronic device 110 or the state of the wearable device 120 to the user through the additional device 130. For example, feedback information with respect to a gait state of the user wearing the wearable device 120 may be output through a haptic device, a speaker device, and a display device of the additional device 130.

According to an embodiment, the electronic device 110 may be connected to the server 140 using short-range wireless communication or cellular communication. For example, the server 140 may include a database in which information about a plurality of workout programs to be provided to a user through the wearable device 120 is stored. For example, the server 140 may manage a user account of the user of the electronic device 110 or the wearable device 120. The server 140 may store and manage a workout program performed by the user and a result of performance with respect to the workout program in link with the user account.

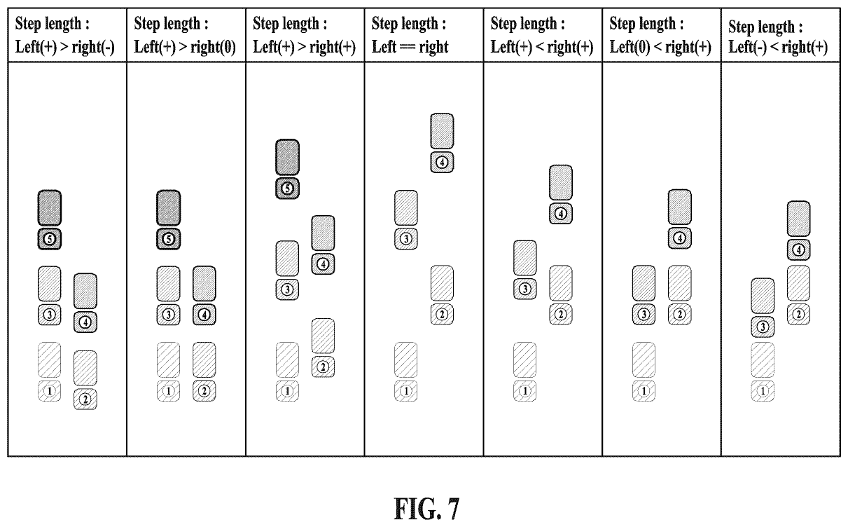

According to an embodiment, the electronic device 100 or the wearable device 120 may determine the gait state of the user wearing the wearable device 120. For example, the gait state of the user may include whether the user has performed a stride motion. The electronic device 100 or the wearable device 120 may increase the number of gait numbers of the user when the user performs the stride motion. As it is accurately determined whether the user performs the stride motion, a counting accuracy of the gait numbers of the user may increase. Hereinafter, the method of determining the gait state of the user will be described in detail with reference to FIGS. 7 to 15.

FIG. 2 is a block diagram of an electronic device in a network environment according to an embodiment.

FIG. 2 is a block diagram of an electronic device 201 (e.g., the electronic device 110 of FIG. 1) in a network environment 200 according to an embodiment. Referring to FIG. 2, the electronic device 201 in the network environment 200 may communicate with an electronic device 202 via a first network 298 (e.g., a short-range wireless communication network), or communicate with at least one of an electronic device 204 or a server 208 via a second network 299 (e.g., a long-range wireless communication network). According to an embodiment, the electronic device 201 may communicate with the electronic device 204 via the server 208. According to an embodiment, the electronic device 201 may include a processor 220, a memory 230, an input module 250, a sound output module 255, a display module 260, an audio module 270, a sensor module 276, an interface 277, a connecting terminal 278, a haptic module 279, a camera module 280, a power management module 288, a battery 289, a communication module 290, a subscriber identification module (SIM) 296, or an antenna module 297. In some embodiments, at least one (e.g., the connecting terminal 278) of the above components may be omitted from the electronic device 201, or one or more other components may be added in the electronic device 201. In some embodiments, some (e.g., the sensor module 276, the camera module 280, or the antenna module 297) of the components may be integrated as a single component (e.g., the display module 260).

The processor 220 may execute, for example, software (e.g., a program 240) to control at least one other component (e.g., a hardware or software component) of the electronic device 201 connected, directly or indirectly, to the processor 220, and may perform various data processing or computation. According to an embodiment, as at least a portion of data processing or computation, the processor 220 may store a command or data received from another component (e.g., the sensor module 276 or the communication module 290) in a volatile memory 232, process the command or the data stored in the volatile memory 232, and store resulting data in a non-volatile memory 234. According to an embodiment, the processor 220 may include a main processor 221 (e.g., a central processing unit (CPU) or an application processor (AP)) or an auxiliary processor 223 (e.g., a graphics processing unit (GPU), a neural processing unit (NPU), an image signal processor (ISP), a sensor hub processor, or a communication processor (CP)) that is operable independently of, or in conjunction with the main processor 221. For example, when the electronic device 201 includes the main processor 221 and the auxiliary processor 223, the auxiliary processor 223 may be adapted to consume less power than the main processor 221 or to be specific to a specified function. The auxiliary processor 223 may be implemented separately from the main processor 221 or as a portion of the main processor 221.

The auxiliary processor 223 may control at least some of functions or states related to at least one (e.g., the display module 260, the sensor module 276, or the communication module 290) of the components of the electronic device 201, instead of the main processor 221 while the main processor 221 is in an inactive (e.g., sleep) state, or together with the main processor 221 while the main processor 221 is in an active state (e.g., executing an application). According to an embodiment, the auxiliary processor 223 (e.g., an ISP or a CP) may be implemented as a portion of another component (e.g., the camera module 280 or the communication module 290) that is functionally related to the auxiliary processor 223. According to an embodiment, the auxiliary processor 223 (e.g., an NPU) may include a hardware structure specified for artificial intelligence (AI) model processing. An AI model may be generated by machine learning. Such learning may be performed, for example, by the electronic device 201 in which an AI mode is executed, or via a separate server (e.g., the server 208). Learning algorithms may include, but are not limited to, for example, supervised learning, unsupervised learning, semi-supervised learning, or reinforcement learning. The AI model may include a plurality of artificial neural network layers. An artificial neural network may include, for example, a deep neural network (DNN), a convolutional neural network (CNN), a recurrent neural network (RNN), a restricted Boltzmann machine (RBM), a deep belief network (DBN), and a bidirectional recurrent deep neural network (BRDNN), a deep Q-network, or a combination of two or more thereof, but is not limited thereto. The AI model may additionally or alternatively include a software structure other than the hardware structure.

The memory 230 may store various pieces of data used by at least one component (e.g., the processor 220 or the sensor module 276) of the electronic device 201. The various pieces of data may include, for example, software (e.g., the program 240) and input data or output data for a command related thereto. The memory 230 may include the volatile memory 232 or the non-volatile memory 234.

The program 240 may be stored as software in the memory 230, and may include, for example, an operating system (OS) 242, middleware 244, or an application 246. The input module 250 may receive a command or data to be used by another component (e.g., the processor 220) of the electronic device 201, from the outside (e.g., a user) of the electronic device 201. The input module 250 may include, for example, a microphone, a mouse, a keyboard, a key (e.g., a button), or a digital pen (e.g., a stylus pen).

The sound output module 255 may output a sound signal to the outside of the electronic device 201. The sound output module 255 may include, for example, a speaker or a receiver. The speaker may be used for general purposes, such as playing multimedia or playing record. The receiver may be used to receive an incoming call. According to an embodiment, the receiver may be implemented separately from the speaker or as a portion of the speaker.

The display module 260 may visually provide information to the outside (e.g., a user) of the electronic device 201. The display module 260 may include, for example, a display, a hologram device, or a projector and control circuitry to control a corresponding one of the display, hologram device, and projector. According to an embodiment, the display module 260 may include a touch sensor adapted to sense a touch, or a pressure sensor adapted to measure an intensity of a force incurred by the touch.

The audio module 270 may convert a sound into an electrical signal or vice versa. According to an embodiment, the audio module 270 may obtain the sound via the input module 250 or output the sound via the sound output module 255 or an external electronic device (e.g., the electronic device 202 such as a speaker or a headphone) directly or wirelessly connected to the electronic device 201.

The sensor module 276 may detect an operational state (e.g., power or temperature) of the electronic device 201 or an environmental state (e.g., a state of a user) external to the electronic device 201, and generate an electrical signal or data value corresponding to the detected state. According to an embodiment, the sensor module 276 may include, for example, a gesture sensor, a gyro sensor, an atmospheric pressure sensor, a magnetic sensor, an acceleration sensor, a grip sensor, a proximity sensor, a color sensor, an infrared (IR) sensor, a biometric sensor, a temperature sensor, a humidity sensor, or an illuminance sensor.

The interface 277 may support one or more specified protocols to be used for the electronic device 201 to be coupled with the external electronic device (e.g., the electronic device 202) directly (e.g., by wire) or wirelessly. According to an embodiment, the interface 277 may include, for example, a high-definition multimedia interface (HDMI), a universal serial bus (USB) interface, a secure digital (SD) card interface, or an audio interface.

The connecting terminal 278 may include a connector via which the electronic device 201 may be physically connected, directly or indirectly, to an external electronic device (e.g., the electronic device 202). According to an embodiment, the connecting terminal 278 may include, for example, an HDMI connector, a USB connector, an SD card connector, or an audio connector (e.g., a headphone connector).

The haptic module 279 may convert an electrical signal into a mechanical stimulus (e.g., a vibration or a movement) or an electrical stimulus which may be recognized by a user via his or her tactile sensation or kinesthetic sensation. According to an embodiment, the haptic module 279 may include, for example, a motor, a piezoelectric element, or an electric stimulator.

The camera module 280 may capture a still image and a video. According to an embodiment, the camera module 280 may include one or more lenses, image sensors, ISPs, or flashes.

The power management module 288 may manage power supplied to the electronic device 201. According to an embodiment, the power management module 288 may be implemented as, for example, at least a portion of a power management integrated circuit (PMIC).

The battery 289 may supply power to at least one component of the electronic device 201. According to an embodiment, the battery 289 may include, for example, a primary cell which is not rechargeable, a secondary cell which is rechargeable, or a fuel cell.

The communication module 290 may support establishing a direct (e.g., wired) communication channel or a wireless communication channel between the electronic device 201 and the external electronic device (e.g., the electronic device 202, the electronic device 204, or the server 208) and performing communication via the established communication channel. The communication module 290 may include one or more communication processors that operate independently of the processor 220 (e.g., an application processor) and support direct (e.g., wired) communication or wireless communication. According to an embodiment, the communication module 290 may include a wireless communication module 292 (e.g., a cellular communication module, a short-range wireless communication module, or a global navigation satellite system (GNSS) communication module) or a wired communication module 294 (e.g., a local area network (LAN) communication module, or a power line communication (PLC) module). A corresponding one of these communication modules may communicate with the external electronic device 204 via the first network 298 (e.g., a short-range communication network, such as Bluetooth™, wireless-fidelity (Wi-Fi) direct, or infrared data association (IrDA)) or the second network 299 (e.g., a long-range communication network, such as a legacy cellular network, a 5G network, a next-generation communication network, the Internet, or a computer network (e.g., a LAN or a wide area network (WAN))). These various types of communication modules may be implemented as a single component (e.g., a single chip), or may be implemented as multi components (e.g., multiple chips) separate from each other. The wireless communication module 292 may identify or authenticate the electronic device 201 in a communication network, such as the first network 298 or the second network 299, using subscriber information (e.g., international mobile subscriber identity (IMSI)) stored in the SIM 296.

The wireless communication module 292, comprising communication circuitry, may support a 5G network after a 4G network, and a next-generation communication technology, e.g., a new radio (NR) access technology. The NR access technology may support enhanced mobile broadband (eMBB), massive machine type communications (mMTC), or ultra-reliable and low-latency communications (URLLC). The wireless communication module 292 may support a high-frequency band (e.g., a mmWave band) to achieve, e.g., a high data transmission rate. The wireless communication module 292 may support various technologies for securing performance on a high-frequency band, such as, e.g., beamforming, massive multiple-input and multiple-output (massive MIMO), full dimensional MIMO (FD-MIMO), an array antenna, analog beamforming, or a large scale antenna. The wireless communication module 292 may support various requirements specified in the electronic device 201, an external electronic device (e.g., the electronic device 204), or a network system (e.g., the second network 299). According to an embodiment, the wireless communication module 292 may support a peak data rate (e.g., 20 Gbps or more) for implementing eMBB, loss coverage (e.g., 164 dB or less) for implementing mMTC, or U-plane latency (e.g., 0.5 ms or less for each of downlink (DL) and uplink (UL), or a round trip of 1 ms or less) for implementing URLLC.

The antenna module 297 may transmit or receive a signal or power to or from the outside (e.g., an external electronic device) of the electronic device 201. According to an embodiment, the antenna module 297 may include an antenna including a radiating element including a conductive material or a conductive pattern formed in or on a substrate (e.g., a printed circuit board (PCB)). According to an embodiment, the antenna module 297 may include a plurality of antennas (e.g., array antennas). In such a case, at least one antenna appropriate for a communication scheme used in a communication network, such as the first network 298 or the second network 299, may be selected by, for example, the communication module 290 from the plurality of antennas. The signal or the power may be transmitted or received between the communication module 290 and the external electronic device via the at least one selected antenna. According to an embodiment, another component (e.g., a radio frequency integrated circuit (RFIC)) other than the radiating element may be additionally formed as a portion of the antenna module 297.

According to an embodiment, the antenna module 297 may form a mm Wave antenna module. For example, the mmWave antenna module may include a PCB, an RFIC disposed on a first surface (e.g., the bottom surface) of the PCB or adjacent to the first surface and capable of supporting a designated high-frequency band (e.g., the mmWave band), and a plurality of antennas (e.g., array antennas) disposed on a second surface (e.g., the top or a side surface) of the PCB, or adjacent to the second surface and capable of transmitting or receiving signals in the designated high-frequency band.

At least some of the above-described components may be coupled mutually and communicate signals (e.g., commands or data) therebetween via an inter-peripheral communication scheme (e.g., a bus, general purpose input and output (GPIO), serial peripheral interface (SPI), or mobile industry processor interface (MIPI)).

According to an embodiment, commands or data may be transmitted or received between the electronic device 201 and the external electronic device 204 via the server 208 coupled with the second network 299. Each of the external electronic devices 202 and 204 may be a device of the same type as or a different type from the electronic device 201. According to an embodiment, all or some of operations to be executed at the electronic device 201 may be executed at one or more of external electronic devices (e.g., the external electronic devices 202 and 204, or the server 208). For example, if the electronic device 201 needs to perform a function or a service automatically, or in response to a request from a user or another device, the electronic device 201, instead of, or in addition to, executing the function or the service, may request one or more external electronic devices to perform at least a part of the function or the service. The one or more external electronic devices receiving the request may perform the at least part of the function or the service requested, or an additional function or an additional service related to the request, and transfer an outcome of the performing to the electronic device 201. The electronic device 201 may provide the outcome, with or without further processing of the outcome, as at least part of a reply to the request. To that end, a cloud computing, distributed computing, mobile edge computing (MEC), or client-server computing technology may be used, for example. The electronic device 201 may provide ultra low-latency services using, e.g., distributed computing or MEC. In another embodiment, the external electronic device 204 may include an Internet-of-things (IoT) device. The server 208 may be an intelligent server using machine learning and/or a neural network. According to an embodiment, the external electronic device 204 or the server 208 may be included in the second network 299. The electronic device 201 may be applied to intelligent services (e.g., a smart home, a smart city, a smart car, or healthcare) based on 5G communication technology or IoT-related technology.

The electronic device according to various embodiments may be one of various types of electronic devices. The electronic device may include, for example, a portable communication device (e.g., a smart phone), a computer device, a portable multimedia device, a portable medical device, a camera, a wearable device, or a home appliance device. According to an embodiment of the disclosure, the electronic device is not limited to those described above.

It should be appreciated that various embodiments of the present disclosure and the terms used therein are not intended to limit the technological features set forth herein to particular embodiments and include various changes, equivalents, or replacements for a corresponding embodiment. In connection with the description of the drawings, like reference numerals may be used for similar or related components. It is to be understood that a singular form of a noun corresponding to an item may include one or more of the things, unless the relevant context clearly indicates otherwise. As used herein, “A or B,” “at least one of A and B,” “at least one of A or B,” “A, B or C,” “at least one of A, B and C,” and “at least one of A, B, or C,” each of which may include any one of the items listed together in the corresponding one of the phrases, or all possible combinations thereof. Terms, such as “first” or “second”, are simply used to distinguish a component from another component and do not limit the components in other aspects (e.g., importance or sequence). It is to be understood that if a component (e.g., a first component) is referred to, with or without the term “operatively” or “communicatively”, as “coupled with,” “coupled to,” “connected with,” or “connected to” another component (e.g., a second component), the component may be coupled with the other component directly (e.g., by wire), wirelessly, or via at least a third component(s).

As used in connection with an embodiment of the disclosure, the term “module” may include a unit implemented in hardware, software, or firmware, and may interchangeably be used with other terms, for example, “logic,” “logic block,” “part,” or “circuitry”. A module may be a single integral component, or a minimum unit or part thereof, adapted to perform one or more functions. For example, according to an embodiment, the module may be implemented in a form of an application-specific integrated circuit (ASIC). Thus, each “module” herein may comprise circuitry.

Various embodiments as set forth herein may be implemented as software (e.g., the program 240) including one or more instructions that are stored in a storage medium (e.g., an internal memory 236 or an external memory 238) that is readable by a machine (e.g., the electronic device 201). For example, a processor (e.g., the processor 220) of the machine (e.g., the electronic device 201) may invoke at least one of the one or more instructions stored in the storage medium, and execute it. This allows the machine to be operated to perform at least one function according to the at least one instruction invoked. The one or more instructions may include code generated by a compiler or code executable by an interpreter. The machine-readable storage medium may be provided in the form of a non-transitory storage medium. Here, the term “non-transitory” simply means that the storage medium is a tangible device, and does not include a signal (e.g., an electromagnetic wave), but this term does not differentiate between where data is semi-permanently stored in the storage medium and where the data is temporarily stored in the storage medium.

According to an embodiment, a method according to various embodiments of the disclosure may be included and provided in a computer program product. The computer program product may be traded as a product between a seller and a buyer. The computer program product may be distributed in the form of a machine-readable storage medium (e.g., compact disc read-only memory (CD-ROM)), or be distributed (e.g., downloaded or uploaded) online via an application store (e.g., PlayStore™), or between two user devices (e.g., smartphones) directly. If distributed online, at least part of the computer program product may be temporarily generated or at least temporarily stored in the machine-readable storage medium, such as memory of the manufacturer's server, a server of the application store, or a relay server.

According to an embodiment, each component (e.g., a module or a program) of the above-described components may include a single entity or multiple entities, and some of the multiple entities may be separately disposed in different components. According to various embodiments, one or more of the above-described components or operations may be omitted, or one or more other components or operations may be added. Alternatively or additionally, a plurality of components (e.g., modules or programs) may be integrated into a single component. In such a case, the integrated component may still perform one or more functions of each of the plurality of components in the same or similar manner as they are performed by a corresponding one of the plurality of components before the integration. According to an embodiment, operations performed by the module, the program, or another component may be carried out sequentially, in parallel, repeatedly, or heuristically, or one or more of the operations may be executed in a different order or omitted, or one or more other operations may be added.

FIGS. 3A, 3B, 3C, and 3D are diagrams illustrating a wearable device according to an embodiment.

Referring to FIGS. 3A, 3B, 3C, and 3D, a wearable device 300 (e.g., the wearable device 120 of FIG. 1) may be worn by a user to assist a gait of the user. For example, the wearable device 300 may be a device for assisting the gait of the user. Further, the wearable device 300 may be a workout device that provides a workout function by assisting a motion (e.g., a gait or a workout) of the user and providing the user with a resistance force. For example, the resistance force provided to the user may be a force actively applied to the user, such as a force output by a device such as a motor. Alternatively, the resistance force may not be a force actively applied to the user, but may be a force that impedes a motion of the user, such as a frictional force. The resistance force may also be referred to as a workout load.

Although FIGS. 3A, 3B, 3C, and 3D illustrate a hip-type wearable device 300, the type of the wearable device is not limited thereto. The wearable device may be a type that supports the entire lower limbs or a type that supports a portion of the lower limbs. In addition, the wearable device may be one of a type that supports a portion of the lower limbs, a type that supports up to the knees, a type that supports up to the ankles, and a type that supports the entire body.

The embodiments described with reference to FIGS. 3A, 3B, 3C, and 3D may apply to a hip-type wearable device, but are not limited thereto, and may all apply to various types of wearable devices.

According to an embodiment, the wearable device 300 may include a driver 310, a sensor unit 320, an inertial measurement unit (IMU) 330, a controller 340 comprising processing circuitry, a battery 350, and a communication module 352. For example, the IMU 330 and the controller 340 may be disposed in a main frame of the wearable device 300. For example, the IMU 330 and the controller 340 may be included in a housing that is formed in (or attached to) the outside of the main frame of the wearable device 300.

The driver 310 may include a motor 314 and a motor driver circuit 312 for driving the motor 314. The sensor unit 320 may include at least one sensor 321. The controller 340 may include a processor 342, a memory 344, and an input interface 346. The memory 344 may comprise one or more storage media storing instructions. The instructions, when executed by at least one processor (e.g., a communication processor) individually or collectively, may cause the electronic device 800 to perform operations described herein. For example, the instructions, when executed by the at least one processor (e.g., a communication processor) individually or collectively, may cause the electronic device 800 to perform at least portion of operations 1010 to 1030, 1110 and 1120, 1210 and 1220, 1310 and 1320, 1410 to 1430, 1510 to 1530, 1610 and 1620, 1710 to 1730. Although one sensor 321, one motor driver circuit 312, and one motor 314 are shown in FIG. 3C, this is merely an example. As in another example shown in FIG. 3D, a wearable device 300-1 may include a plurality of sensors 321 and 321-1, a plurality of motor driver circuits 312 and 312-1, and a plurality of motors 314 and 314-1. Also, according to implementation, the wearable device 300 may include a plurality of processors. The number of motor driver circuits, the number of motors, or the number of processors may vary depending on a body part on which the wearable device 300 is worn. Each “processor” herein includes processing circuitry, and/or may include multiple processors. For example, as used herein, including the claims, the term “processor” may include various processing circuitry, including at least one processor, wherein one or more of at least one processor, individually and/or collectively in a distributed manner, may be configured to perform various functions described herein. As used herein, when “a processor”, “at least one processor”, and “one or more processors” are described as being configured to perform numerous functions, these terms cover situations, for example and without limitation, in which one processor performs some of recited functions and another processor(s) performs other of recited functions, and also situations in which a single processor may perform all recited functions. Additionally, the at least one processor may include a combination of processors performing various of the recited/disclosed functions, e.g., in a distributed manner. At least one processor may execute program instructions to achieve or perform various functions.

The following description of the sensor 321, the motor driver circuit 312, and the motor 314 may also apply to the sensor 321-1, the motor driver circuit 312-1, and the motor 314-1 illustrated in FIG. 3D.

The driver 310 may drive a hip joint of a user. For example, the driver 310 may be positioned on the right hip portion and/or the left hip portion of the user. The driver 310 may be additionally positioned on the knee portions and the ankle portions of the user. The driver 310 may include the motor 314 for generating a rotational torque and the motor driver circuit 312 for driving the motor 314.

The sensor unit 320 may measure the angles of the hip joints of the user during a gait. Information on the angles of the hip joints sensed by the sensor unit 320 may include the angle of the right hip joint, the angle of the left hip joint, the difference between the angles of both hip joints, and the hip joint motion direction. For example, the sensor 321 may be positioned in the driver 310. According to the position of the sensor 321, the sensor unit 320 may additionally measure the angles of the knees and the angles of the ankles of the user. The sensor 321 may be, for example, an encoder. The sensor 321 may be, for example, a Hall sensor. The information on the angles of the joints measured by the sensor unit 320 may be transmitted to the controller 340.

According to an embodiment, the sensor unit 320 may include a potentiometer. The potentiometer may sense an R-axis joint angle, an L-axis joint angle, an R-axis joint angular velocity, and an L-axis joint angular velocity according to a gait motion of the user. The R/L axis may be a reference axis for the right/left leg of the user. For example, the R/L axis may be set to be vertical to the ground and set such that a front side of a body of a person has a negative value and a rear side of the body has a positive value.

The IMU 330 may measure acceleration information and pose information during a gait. For example, the IMU 330 may sense X-axis, Y-axis, and Z-axis accelerations and X-axis, Y-axis, and Z-axis angular velocities according to the gait motion of the user. The acceleration information and pose information measured by the IMU 330 may be transmitted to the controller 340.

In addition to the sensor unit 320 and the IMU 330 described above, the wearable device 300 may include a sensor (e.g., an electromyogram (EMG) sensor) configured to sense a change in a quantity of motion of the user or a change in a biosignal according to a gait motion.

The controller 340 may control an overall operation of the wearable device 300. For example, the controller 340 may receive the information sensed by each of the sensor unit 320 and the IMU 330. The information sensed by the IMU 330 may include acceleration information and pose information, and the information sensed by the sensor unit 320 may include the angle of the right hip joint, the angle of the left hip joint, the difference between the angles of the two hip joints, and the hip joint motion direction. According to an embodiment, the controller 340 may calculate the difference between the angles of both hip joints based on the angle of the right hip joint and the angle of the left hip joint. The controller 340 may generate a signal for controlling the driver 310 based on the sensed information. For example, the generated signal may be an assistance force for assisting a motion of the user. Alternatively, the generated signal may be a resistance force for impeding a motion of the user. The resistance force may be provided to assist the user in doing a workout. In the following description, a negative magnitude of a workout load (or a torque) may indicate a resistance force, and a positive magnitude thereof may indicate an assistance force.

According to an embodiment, the processor 342 of the controller 340 may control the driver 310 to provide the user with a resistance force. For example, the driver 310 may provide the user with a resistance force by applying an active force to the user through the motor 314. Alternatively, the driver 310 may provide the user with a resistance force using the back-drivability of the motor 314, without applying an active force to the user. The back-drivability of the motor may be a responsiveness of the rotation axis of the motor to an external force. When the back-drivability of the motor increases, the motor may more readily respond to an external force acting on the rotation axis of the motor (that is, the rotation axis of the motor may more readily rotate). Even when the same external force is applied to the rotation axis of the motor, the degree of rotation of the rotation axis of the motor may vary depending on the degree of back-drivability.

According to an embodiment, the processor 342 of the controller 340 may control the driver 310 to output a torque (or an assisting torque) for assisting a motion of the user. For example, in the hip-type wearable device 300, the driver 310 may be disposed on each of the left hip portion and the right hip portion, and the controller 340 may output a control signal for controlling the driver 310 to generate a torque.

The driver 310 may generate a torque based on the control signal output by the controller 340. A torque value for generating the torque may be externally set or be set by the controller 340. For example, to indicate a magnitude of the torque value, the controller 340 may use a magnitude of a current for the signal transmitted to the driver 310. That is, as the magnitude of the current received by the driver 310 increases, the torque value may increase. As another example, the processor 342 of the controller 340 may transmit the control signal to the motor driver circuit 312 of the driver 310, and the motor driver circuit 312 may generate a current corresponding to the control signal to control the motor 314.

The battery 350 may supply power to the components of the wearable device 300. The wearable device 300 may further include a circuit (e.g., a PMIC) configured to convert the power of the battery 350 according to an operating voltage of the components of the wearable device 300 and provide the same to the components of the wearable device 300. In addition, the battery 350 may or may not supply power to the motor 314 based on an operation mode of the wearable device 300.

The communication module 352 may support the establishment of a direct (or wired) communication channel or a wireless communication channel between the wearable device 300 and an external electronic device, and support the communication through the established communication channel. The communication module 352 may include one or more communication processors configured to support direct (or wired) communication or wireless communication. According to an embodiment, the communication module 352 may include a wireless communication module (e.g., a cellular communication module, a short-range wireless communication module, or a global navigation satellite system (GNSS) communication module) or a wired communication module (e.g., a local area network (LAN) communication module or a power line communication (PLC) module). A corresponding one of these communication modules may communicate with the external electronic device via a first network (e.g., a short-range communication network such as Bluetooth™, Wi-Fi direct, or IrDA) or a second network (e.g., a legacy cellular network, a 5G network, a next-generation communication network, the Internet, or a computer network). These various types of communication modules may be implemented as a single component (e.g., a single chip), or may be implemented as multi components (e.g., multiple chips) separate from each other.

According to an embodiment, the electronic device 201 described above with reference to FIG. 2 may be included in the wearable device 300.

According to an embodiment, the electronic device 201 described above with reference to FIG. 2 may be a separate device physically separated from the wearable device 300, and the electronic device 201 and the wearable device 300 may be connected through short-range wireless communication.

FIG. 4 is a diagram illustrating a wearable device communicating with an electronic device according to an embodiment.

Referring to FIG. 4, the wearable device 300 (e.g., the wearable device 120 of FIG. 1) described above with reference to FIGS. 3A, 3B, 3C, and 3D may communicate with the electronic device 201 (e.g., the electronic device 110 of FIG. 1) described above with reference to FIG. 2. For example, the electronic device 201 may be an electronic device of a user of the wearable device 300. According to an embodiment, the wearable device 300 and the electronic device 201 may be connected using a short-range wireless communication method.

The electronic device 201 may display a user interface (UI) for controlling an operation of the wearable device 300 on a display 201-1. The UI may include, for example, at least one soft key through which the user may control the wearable device 300.

The user may input a command for controlling the operation of the wearable device 300 through the UI on the display 201-1 of the electronic device 201, and the electronic device 201 may generate a control instruction corresponding to the command and transmit the generated control instruction to the wearable device 300. The wearable device 300 may operate according to the received control instruction, and transmit a control result to the electronic device 201. The electronic device 201 may display a control completion message on the display 201-1 of the electronic device 201.

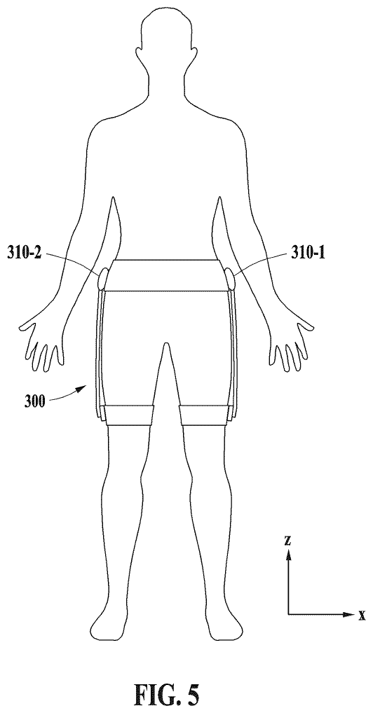

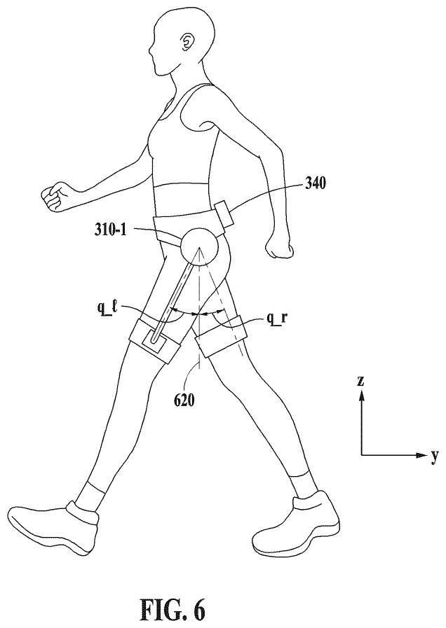

FIGS. 5 and 6 are diagrams illustrating a torque output method of a wearable device according to an embodiment.

Referring to FIGS. 5 and 6, drivers 310-1 and 310-2 of the wearable device 300 of FIG. 3 (e.g., the wearable device 120 of FIG. 1) may be disposed near the hip joints of a user, and the controller 340 of the wearable device 300 may be disposed near the lower back of the user. The positions of the drivers 310-1 and 310-2 and the controller 340 are not limited to the example positions illustrated in FIGS. 5 and 6.

The wearable device 300 may measure (or sense) a left hip joint angle q_l and a right hip joint angle q_r of the user. For example, the wearable device 300 may measure the left hip joint angle q_l of the user through a left encoder (or a Hall sensor), and measure the right hip joint angle q_r of the user through a right encoder. As illustrated in FIG. 6, the left hip joint angle q_l may be negative because the left leg of the user is in front of a reference line 620, and the right hip joint angle q_r may be positive because the right leg of the user is behind the reference line 620. According to an implementation example, the right hip joint angle q_r may be negative when the right leg is in front of the reference line 620, and the left hip joint angle q/may be positive when the left leg is behind the reference line 620.

According to an embodiment, the wearable device 300 may obtain a first angle (e.g., q_r) and a second angle (e.g., q_l) by filtering a first raw angle (e.g., q_r_raw) of a first joint (e.g., the right hip joint) and a second raw angle (e.g., q_l_raw) of a second joint (e.g., the left hip joint) measured by the sensor unit 320. For example, the wearable device 300 may filter the first raw angle and the second raw angle based on a first previous angle and a second previous angle measured with respect to a previous time.

According to an embodiment, the wearable device 300 may determine a torque value τ(t) based on the left hip joint angle q_l, the right hip joint angle q_r, an offset angle c, a sensitivity a, a gain K, and a delay Δt, and control the motor driver circuit 312 of the wearable device 300 to output the determined torque value τ(t). The force provided to the user by the torque value τ(t) may be referred to herein as force feedback. For example, the wearable device 300 may determine the torque value τ(t) based on Equation 1 below.

$$y = sin ( q\_r ) - sin ( q\_l ) \qquad [ Equation 1 ]$$

$$τ ( t ) = κ y ( t - Δ t )$$

In Equation 1, y denotes a state factor, q_r denotes the right hip joint angle, and q_l denotes the left hip joint angle. According to Equation 1, the state factor y may be associated with the distance between the two legs. For example, y being “O” may indicate a state (e.g., a crossing state) in which the distance between the legs is “0”, and the absolute value of y being maximum may indicate a state (e.g., a landing state) in which the angle between the legs is maximum. According to an embodiment, when q_r and q_l are measured at a time t, the state factor may be represented as y(t).

The gain κ is a parameter indicating the magnitude and direction of an output torque. As the magnitude of the gain κ increases, a greater torque may be output. If the gain κ is negative, a torque acting as a resistance force may be output to the user, and if the gain κ is positive, a torque acting as an assistance force may be output to the user. The delay Δt is a parameter associated with a torque output timing. The value of the gain k and the value of the delay Δt may be preset, and may be adjustable by a user, the wearable device 300, or the electronic device 201 described above with reference to FIG. 2.

A model for outputting a torque acting as an assistance force to a user using Equation 1 may be a torque output model (or a torque output algorithm). The wearable device 300 or the electronic device 201 may determine the magnitude and delay of a torque to be output by inputting the values of input parameters received through sensors into the torque output model.

According to an embodiment, the wearable device 300 or the electronic device 201 may determine a first torque value through Equation 2 below by applying a first gain value and a first delay value to a first state factor y(t), wherein the first gain value and the first delay value may be parameter values determined with respect to the state factor y(t).

$${τ}_{l} ( t ) = κ y ( t - Δ t ) \qquad [ Equation 2 ]$$

$${τ}_{r} ( t ) = - κ y ( t - Δ t )$$

The calculated first torque value may include a value for the first joint and a value for the second joint since it should be applied to the two legs. For example, τl(t) may be a value for the left hip joint, which is the second joint, and τr(t) may be a value for the right hip joint, which is the first joint. τl(t) and τr(t) may be values with the same magnitude and opposite torque directions. The wearable device 300 may control the motor driver circuit 312 of the wearable device 300 to output a torque corresponding to the first torque value.

According to an embodiment, when the user performs an asymmetrical gait with the left leg and the right leg, the wearable device 300 may provide asymmetrical torques respectively to both legs of the user to assist the asymmetric gait. For example, a stronger assistance force may be provided to a leg with a shorter stride width or a slower swing speed. Hereinafter, a leg with a small stride width or a slow swing speed will be referred to as an affected leg or a target leg.

In general, an affected leg may have a shorter swing time or a smaller stride width than an unaffected leg. According to an embodiment, a method of adjusting the timing of a torque acting on an affected leg to assist a gait of a user may be considered. For example, an offset angle may be added to an actual joint angle of an affected leg to increase an output time of a torque for assisting a swing motion of the affected leg. C may be the value of a parameter indicating an offset angle between joint angles. As the offset angle is added to the actual joint angle of the affected leg, the value of an input parameter that is input into the torque output model mounted on (or applied to) the wearable device 300 may be adjusted. For example, the values of q_r and q_l may be adjusted through Equation 3 below. Cᵣ denotes an offset angle with respect to the right hip joint, and cₗ denotes an offset angle with respect to the left hip joint.

$${q}_{- r} ( t ) ← {q}_{- r} ( t ) + {c}_{r} \qquad [ Equation 3 ]$$

$${q}_{- l} ( t ) ← {q}_{- l} ( t ) + {c}_{l}$$

According to an embodiment, the wearable device 300 may filter the state factor to reduce the discomfort the user may experience due to irregular torque outputs. For example, the wearable device 300 or the electronic device 201 may determine an initial state factor y_{raw}(t) of a current time t based on the first angle of the first joint and the second angle of the second joint, and determine the first state factor y(t) based on a previous state factor y^{pre} determined with respect to a previous time t−1 and the initial state factor y_{raw}(t). The current time t may be a time at which t-th data (or sample) is processed, and the previous time t−1 may be a time at which t−1-th data is processed. For example, the difference between the current time t and the previous time t−1 may be an operation interval of a processor for generating or processing the corresponding items of data. The sensitivity a may be the value of a parameter indicating a sensitivity. For example, the sensitivity value may be continuously adjusted during a test gait. However, the sensitivity value may be preset to a predetermined value to reduce the computational complexity.

According to an embodiment, a torque output method based on the state factor described with reference to Equation 1 to Equation 3 may be used when the user wearing the wearable device 300 is in a walking state. When the user is in an in-place workout state rather than a walking state, a motion control model corresponding to the workout performed by the user may be used to control the wearable device 300.

FIG. 7 is a diagram illustrating various gaits according to an embodiment.

Referring to FIG. 7, a normal or abnormal gait may occur when a user walks. For example, an abnormal gait may occur when the left and right legs do not cross each other when walking. For example, the abnormal gait may occur when a user performs a normal gait but turns 90 to 180 degrees around a direction of the body.

According to an embodiment, the normal gait and the abnormal gait may be distinguished by whether the left and right legs cross. The crossing state refers to a state in which a sign indicating a distance between the legs changes based on a state in which the distance is 0 (or a state in which a size of an internal angle of the legs is 0). Whether the left and right legs cross may be determined by measuring a left step length and a right step length. For example, a step length may be the distance from a position of the left (or right) heel to a position of the right (or left) heel immediately behind during walking. When the left step length is positive (+) and the right step length is negative (−), the two legs do not cross. When the left step length is positive (+) and the right step length is 0, the two legs cross. When both the left step length and the right step length are positive (+) and the left step length is greater, the two legs cross. When the left step length and the right step length are equal, the legs cross. In the above description, the same applies even when the left and right are exchanged. When the legs cross while the user is walking, it may be considered as the normal gait.

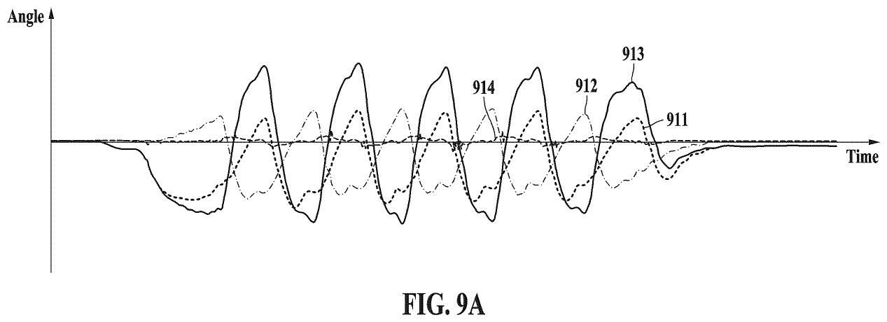

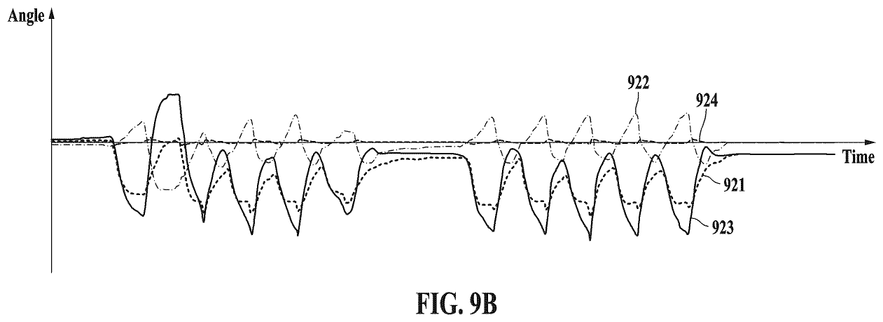

In order to analyze the gait of the user or determine the number of steps, it is necessary to detect the gait state, including a gait cycle. Depending on various gaits of the normal and abnormal gait, detected patterns of gait states may appear differently. This will be described in detail with reference to FIGS. 9A and 9B below.

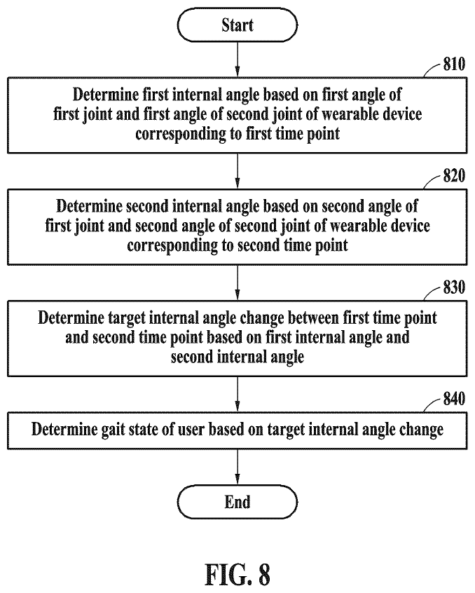

FIG. 8 is a flowchart of a method of determining a gait state according to an embodiment.

According to an embodiment, an electronic device (e.g., the electronic device 110 of FIG. 1 or the electronic device 201 of FIG. 2) may obtain information measured (or sensed) by a wearable device (e.g., the wearable device 120 of FIG. 1 or the wearable device 300 of FIG. 3) from the wearable device. For example, the electronic device may obtain an angle of a first joint (e.g., a right hip joint) and an angle of a second joint (e.g., a left hip joint) from the wearable device.

According to an embodiment, the electronic device (e.g., the wearable device 120 of FIG. 1 or the wearable device 300 of FIG. 3) may obtain information measured by the wearable device. For example, the electronic device may obtain information measured by a sensor unit (e.g., the sensor unit 320 of FIG. 3) or an IMU (e.g., the IMU 330 of FIG. 3) of the wearable device. For example, the electronic device may obtain the angle of the first joint and the angle of the second joint measured by the sensor unit of the wearable device.

According to an embodiment, operations 810 to 840 of FIG. 8 may be performed by an electronic device (e.g., the electronic device 110 of FIG. 1, the electronic device 201 of FIG. 2, the wearable device 120 of FIG. 1, or the wearable device 300 of FIG. 3).

According to an embodiment, the electronic device may obtain a first angle of the first joint and a first angle of the second joint at a first time point. The electronic device may obtain the second angle of the first joint and the second angle of the second joint at a second time point. The electronic device may continuously obtain angles of the first joint and the second joint, and the first time point, the second time point, and the first angle and the second angle corresponding to individual time points are merely examples and are not limited thereto.

In operation 810, the electronic device may determine a first internal angle based on a first angle of a first joint and a first angle of a second joint of a wearable device corresponding to a first time point. The first internal angle may be determined by calculating a difference between the first angle of the second joint and the first angle of the first joint. For example, the first internal angle may be determined by calculating a difference between the first angle of a left hip joint and the first angle of a right hip joint.

In operation 820, the electronic device may determine a second internal angle based on a second angle of the first joint and a second angle of the second joint of the wearable device corresponding to a second time point. The second internal angle may be determined by calculating a difference between the second angle of the second joint and the second angle of the first joint. For example, the first internal angle may be determined by calculating a difference between the second angle of the left hip joint and the second angle of the right hip joint.

In operation 830, the electronic device may determine a target internal angle change between the first time point and the second time point based on the first internal angle and the second internal angle. The target internal angle change may be determined by calculating the difference between the second internal angle and the first internal angle. The target internal angle change may be continuously determined in response to the electronic device continuously obtaining the angles of the first joint and the second joint from the wearable device, and the first internal angle and the second internal angle are merely examples and are not limited thereto.

In operation 840, the electronic device may determine a gait state of the user based on the target internal angle change. The gait state of the user may include at least one of a walking state, a running state, an increased number of strides, gait symmetry, a gait cycle, a gait speed, and a gait rhythm. The content for determining the gait state of the user will be described in detail below with reference to FIGS. 9 to 15.

FIG. 9A is a diagram illustrating sensing data of hip joints for a normal gait according to an embodiment.

A graph of FIG. 9A shows a change in sensing data according to the gait of the user. A horizontal axis of the graph indicates time, and a vertical axis indicates a hip joint angle or a change in hip joint angle. A curve 911 may indicate the left hip joint angle, a curve 912 may indicate the right hip joint angle, a curve 913 may indicate a difference between the left hip joint angle and the right hip joint angle, and a curve 914 may indicate a change in the difference between the two hip joint angles. That is, the curve 913 may indicate a trajectory of an internal angle of both hip joints (hereinafter, the internal angle), and the curve 914 may indicate a trajectory of the internal angle change. The points where the curve 913 intersects the horizontal axis may imply that a value of the trajectory of the internal angle is 0, which thus represents the crossing of both legs. As described with reference to FIG. 7, the graph of FIG. 9A corresponds to sensing data for the normal gait.

According to the trajectory (e.g., the curve 913) of the internal angle according to the normal gait of the user, it may be found that the points where the value of the trajectory of the internal angle becomes 0 are repeated, indicating that the stride occurs repeatedly. A stride refers to a process until the position of one leg returns to the same point through the walking. For example, a stride length may be a distance from the position of the left (or right) heel to the position of the next left heel during walking. For example, when the value of the trajectory of the internal angle is changed from a positive number to a negative number at the first time point, it may be determined that a right heel strike event has occurred immediately before the first time point, and thus right heel strike events may also be determined to occur repeatedly at the second time point, the third time point, and the like when the value of the trajectory of the internal angle is changed from a positive number to a negative number after the first time point. The right heel stride events described above may indicate that the stride occurs repeatedly. In a case of the normal gait, it may be determined whether the stride has occurred only from the trajectory of the internal angle, and the number of steps may be determined based on whether the stride has occurred.

FIG. 9B is a diagram illustrating sensing data of hip joints for an abnormal gait according to an embodiment.

A graph of FIG. 9B shows a change in sensing data according to the gait of the user. A horizontal axis of the graph indicates time, and a vertical axis indicates a hip joint angle or a change in hip joint angle. A curve 921 may indicate the left hip joint angle, a curve 922 may indicate the right hip joint angle, a curve 923 may indicate a difference between the left hip joint angle and the right hip joint angle, and a curve 924 may indicate a change in the difference between the two hip joint angles. That is, the curve 923 may indicate a trajectory of an internal angle, and the curve 924 may indicate a trajectory of the internal angle change. Unlike the curve 913 of FIG. 9A, the curve 923 does not intersect the horizontal axis in response to the change of both hip joint angles, which may indicate that both legs do not cross. As described with reference to FIG. 7, the graph of FIG. 9B corresponds to sensing data for the abnormal gait.

According to the trajectory of the internal angle according to the abnormal gait of the user (e.g., the curve 923), it is not possible to know whether the stride occurs because the points where the value of the trajectory of the internal angle becomes 0 are not repeated. According to an embodiment, in the case of the abnormal gait, since it is not possible to determine whether the stride has occurred only from the trajectory of the internal angle, whether the stride has occurred may be determined through the trajectory of the internal angle change (e.g., the curve 924).

According to an embodiment, whether the stride has occurred may be determined based on whether a predetermined stride event has occurred.

For example, a predetermined stride event may be an event in which a value of an internal angle change trajectory is changed from a positive number to a negative number at a first time, and the value of the internal angle change trajectory is changed from a positive number to a negative number at a second time that is after the first time. When it is determined that the predetermined stride event has occurred, an electronic device (e.g., the electronic device 110 and the wearable device 120 of FIG. 1, the electronic device 201 of FIG. 2, or the wearable device 300 of FIG. 3) may determine the number of steps by increasing the number of strides.

For example, the predetermined stride event may be an event in which the value of the internal angle change trajectory is changed from a negative number to a positive number at the first time, and the value of the internal angle change trajectory is changed from a negative number to a positive number at the second time that is after the first time. When it is determined that the predetermined stride event has occurred, the electronic device may determine the number of steps by increasing the number of strides.

For example, it may be determined whether the predetermined stride event has occurred based on a trajectory (e.g., the curve 914) of the internal angle change during the normal gait.

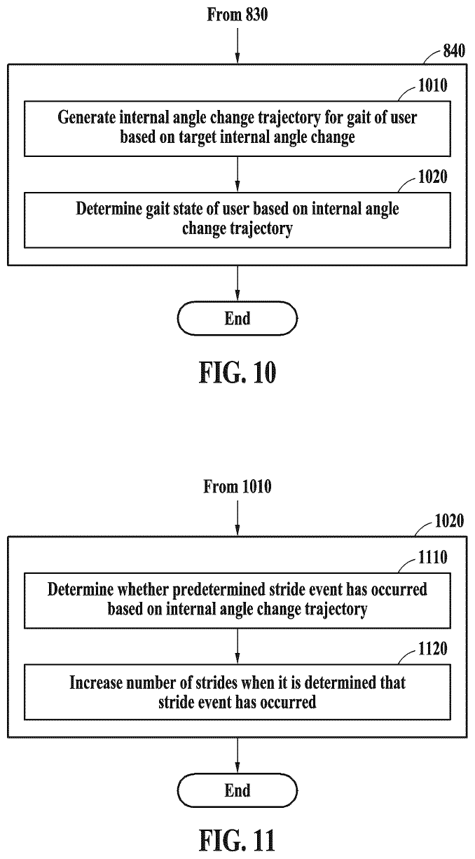

FIG. 10 is a flowchart of a method of determining a gait state of a user based on a target internal angle change according to an example.

According to an embodiment, operation 840 described above with reference to FIG. 8 may include operations 1010 and 1020 below. Operations 1010 and 1020 may be performed by an electronic device (e.g., the electronic device 110 and the wearable device 120 of FIG. 1, the electronic device 201 of FIG. 2, or the wearable device 300 of FIG. 3).

In operation 1010, the electronic device may generate an internal angle change trajectory for a gait of the user based on the target internal angle change. In response to the electronic device continuously obtaining angles of the first joint and the second joint from the wearable device, the electronic device may continuously determine the target internal angle change. For example, the internal angle change trajectory may be the curve 914 of FIG. 9A or the curve 924 of FIG. 9B.

In operation 1020, the electronic device may determine the gait state of the user based on the internal angle change trajectory. For example, the gait state of the user may include a walking state, a running state, an increased number of strides, gait symmetry, a gait cycle, a gait speed, a gait rhythm, or a combination thereof.

FIG. 11 is a flowchart of a method of determining a gait state of a user based on an internal angle change trajectory according to an example.

According to an embodiment, operation 1020 described above with reference to FIG. 10 may include operations 1110 and 1120 below. Operations 1110 and 1120 may be performed by an electronic device (e.g., the electronic device 110 and the wearable device 120 of FIG. 1, the electronic device 201 of FIG. 2, or the wearable device 300 of FIG. 3).

In operation 1110, the electronic device may determine whether a predetermined stride event has occurred based on the internal angle change trajectory. A stride refers to a process until the position of one leg returns to the same point through the walking. For example, a stride length may be a distance from the position of the left (or right) heel to the position of the next left heel during walking.

According to an embodiment, the predetermined stride event may be an event in which the value of the internal angle change trajectory is changed from a positive number to a negative number at the first time, and the value of the internal angle change trajectory is changed from a positive number to a negative number at the second time that is after the first time.

According to an embodiment, the predetermined stride event may be an event in which the value of the internal angle change trajectory is changed from a negative number to a positive number at the first time, and the value of the internal angle change trajectory is changed from a negative number to a positive number at the second time that is after the first time.

In operation 1120, when it is determined that the predetermined stride event has occurred, the electronic device may increase the number of strides. For example, when the predetermined stride event has occurred, the number of steps may be determined by increasing the number of strides.

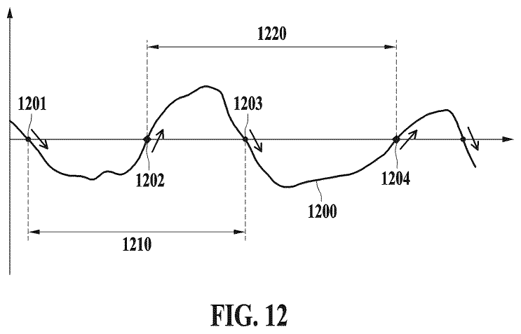

FIG. 12 is a diagram illustrating a predetermined stride event according to an example.

A curve 1200 shown in FIG. 12 may represent a trajectory of the internal angle change.

According to an embodiment, the predetermined stride event may be an event in which the value of the internal angle change trajectory is changed from a positive number to a negative number at a time point 1201 and from a positive number to a negative number at a time point 1203. For example, a gait motion in a section 1210 between the time point 1201 and the time point 1203 may be one stride for the right leg.

According to an embodiment, the predetermined stride event may be an event in which the value of the internal angle change trajectory is changed from a negative number to a positive position at a time point 1202 and from a negative number to a positive position at a time point 1204. For example, the gait motion in a section 1220 between the time point 1202 and the time point 1204 may be one stride for the left leg.

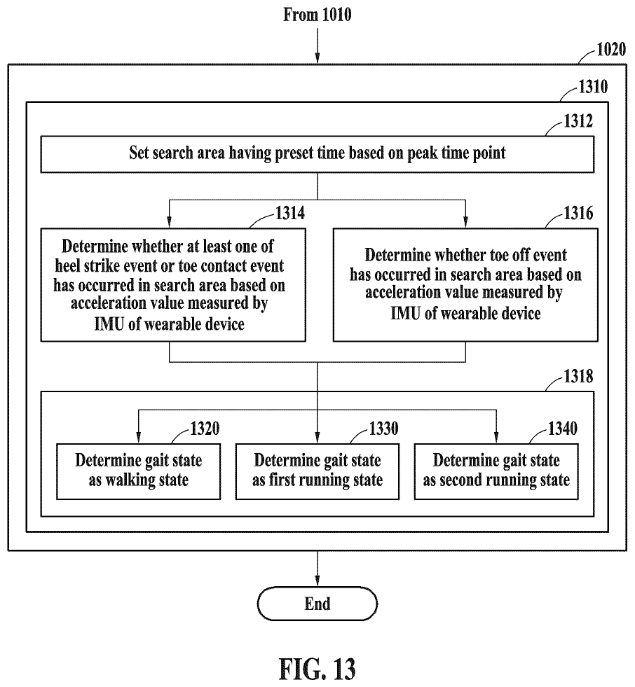

FIG. 13 is a flowchart of a method of determining a gait state of a user based on an internal angle change trajectory according to an example.

According to an embodiment, operation 1020 described above with reference to FIG. 10 may include operation 1310 below. Operation 1310 may be performed by an electronic device (e.g., the electronic device 110 and the wearable device 120 of FIG. 1, the electronic device 201 of FIG. 2, or the wearable device 300 of FIG. 3).

In operation 1310, the electronic device may determine the gait state of the user based on a peak time point at which a value of the internal angle change trajectory is at a peak (e.g., a minimum peak or a maximum peak). For example, the electronic device may determine the gait state of the user based on the peak time point at which the value of the internal angle change trajectory based on the left foot is at the maximum peak (or the value of the internal angle change trajectory based on the right foot is at the minimum peak). For example, the gait state of the user may include a walking state, a running state, an increased number of strides, gait symmetry, a gait cycle, a gait speed, a gait rhythm, or a combination thereof. Hereinafter, the gait state of the user will be described in detail based on the right foot. According to an embodiment, operation 1310 may include operations 1312 to 1318 below.

In operation 1312, the electronic device may set a search area having a preset time based on the peak time point of the internal angle change trajectory. Based on the right foot, the search area may be a section having a preset time between a time point before certain period of time from the peak time point, at which the value of the internal angle change trajectory is at the minimum peak, and a time point after certain period of time from the peak time point. For example, the search area may include the peak time point at which the value of the internal angle change trajectory is at the minimum peak, and may be a section in which the value of the internal angle change trajectory is a negative number (e.g., a section between the time point 1201 and the time point 1202 or the section between the time point 1203 and the time point 1204 of FIG. 12).

In operation 1314, the electronic device may determine whether at least one of a heel strike event or a toe contact event has occurred in the search area based on an acceleration value measured (or sensed) by an IMU (e.g., the IMU 330 of FIG. 3) of the wearable device (e.g., the wearable device 120 of FIG. 1 or the wearable device 300 of FIG. 3). The IMU may sense at least one of X-axis, Y-axis, and Z-axis accelerations or X-axis, Y-axis, and Z-axis angular velocities according to the gait motion of the user.

According to an embodiment, when the number of time points, at which the value of a Z-axis acceleration trajectory sensed by the IMU is at a maximum peak, is 2 in the search area, a first peak may indicate a heel strike event of the right foot, and a second peak may indicate a toe contact event of the right foot. When the number of time points, at which the value of the Z-axis acceleration trajectory sensed by the IMU is at a maximum peak, is 2 in the search area, it may be determined that both the heel strike event and the toe contact event have occurred.

According to an embodiment, when the number of time points, at which the value of the Z-axis acceleration trajectory sensed by the IMU is at a maximum peak, is 1 in the search area, the peak may indicate the heel strike event or the toe contact (or strike) event of the right foot. When the number of time points, at which the value of the Z-axis acceleration trajectory sensed by the IMU is at a maximum peak, is 1 in the search area, it may be determined that one of the heel strike event or the toe contact event has occurred.

In operation 1316, the electronic device may determine whether a toe off event has occurred in the search area based on the acceleration value measured by the IMU of the wearable device.

According to an embodiment, a point at which the value of the X-axis acceleration trajectory sensed by the IMU passes the maximum peak and is changed from a positive number to a negative number (e.g., point at which the value becomes 0) in the search area may indicate the toe off event of the left foot. When the value of the X-axis acceleration trajectory sensed by the IMU passes the maximum peak in the search area and becomes 0, the electronic device may determine that the toe off event has occurred.

In operation 1318, the electronic device may determine the gait state of the user based on the at least one of the heel strike event, the toe contact event, or a combination thereof. According to an embodiment, operation 1318 may include operations 1320 to 1340 below.

According to an embodiment, operation 1318 may further include an operation (not shown) of determining the gait state of the user based on an occurrence time of the heel strike event and an occurrence time of the toe off event, when the heel strike event and the toe contact event have occurred.

According to an embodiment, the operation (not shown) of determining the gait state of the user based on the occurrence time of the heel strike event and the occurrence time of the toe off event, when the heel strike event and the toe contact event have occurred, may include operations 1320 and 1330 below.

In operation 1320, the electronic device may determine the gait state as a walking state, when the occurrence time of the heel strike event precedes the occurrence time of the toe off event. For example, a case where the occurrence time of the heel strike event of one foot precedes the occurrence time of the toe off event of the other foot, may be shown in the walking state where both feet of the user are on the ground at the same time. According to an embodiment, the electronic device may determine the gait state as the walking state when the occurrence time of the heel strike event of the right foot precedes the occurrence time of the toe off event of the left foot.

According to an embodiment, the electronic device may determine the gait state as the walking state when the occurrence time of the heel strike event of the left foot precedes the occurrence time of the toe off event of the right foot.

In operation 1330, the electronic device may determine the gait state as a first running state, when the occurrence time of the heel strike event is later than the occurrence time of the toe off event. For example, a case where the occurrence time of the heel strike event of one foot is later than the occurrence time of the toe off event of the other foot may be shown in the first running where the both feet of the user are not on the ground. For example, the first running may indicate running with a foot landing pattern similar to that of a long-distance run.

According to an embodiment, the electronic device may determine the gait state as the first running state when the occurrence time of the heel strike event of the right foot is later than the occurrence time of the toe off event of the left foot. The time between the occurrence time of the toe off event of the left foot and the occurrence time of the heel strike event of the right foot may indicate a state in which both the left foot and the right foot are in the air, and therefore, the gait state may be determined as the first running state.

According to an embodiment, the electronic device may determine the gait state as the first running state when the occurrence time of the heel strike event of the left foot is later than the occurrence time of the toe off event of the right foot.

In operation 1340, the electronic device may determine the gait state as a second running state, when only the toe contact event has occurred.

For example, a case where only the toe contact event has occurred without the occurrence of the heel strike event or the toe off event may be shown in the second running in which only a part of the user's feet (e.g., the forefoot) touches the ground. For example, the second running may indicate running with a foot landing pattern similar to a short-distance run. For example, the second running state may be a state of running faster than the first running state.

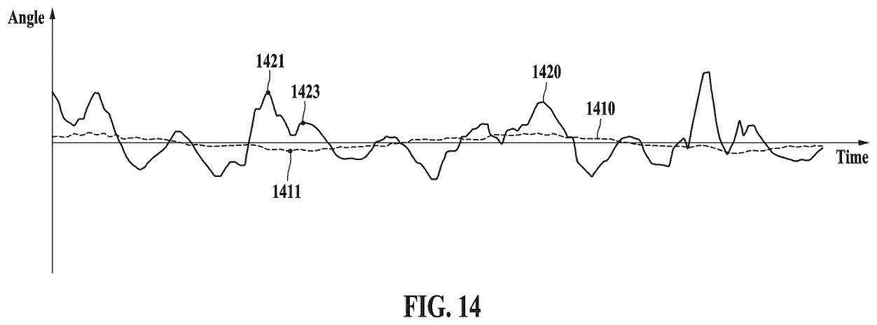

FIG. 14 is a diagram illustrating sensing data for determining a gait state of a user based on a peak time point according to an example.

A graph of FIG. 14 shows a change in sensing data according to the gait of the user. A horizontal axis of the graph indicates time, and a vertical axis indicates a change in hip joint angle or an acceleration measured (or sensed) by an IMU (e.g., the IMU 330 of FIG. 3) of a wearable device (e.g., the wearable device 120 of FIG. 1 or the wearable device 300 of FIG. 3). A curve 1410 may indicate a change in a difference between both hip joint angles, and a curve 1420 may indicate a Z-axis acceleration measured by the IMU of the wearable device. The curve 1410 may indicate a trajectory of the internal angle change. A point 1411 may indicate a peak time point at which the value of the internal angle change trajectory is at a peak, and points 1421 and 1423 may indicate time points at which values of the Z-axis acceleration trajectory sensed by the IMU are at maximum peaks in the search area.

Referring to FIG. 14, the electronic device may determine the gait state of the user based on the peak time point corresponding to the point 1411 at which the value of the internal angle change trajectory is at the minimum peak based on the right foot. The electronic device may set the search area having a preset time based on the peak time point corresponding to the point 1411. For example, the search area may be a section that includes the point 1411 and in which the value of the internal angle change trajectory is negative. For example, when the number of time points, at which the value of the Z-axis acceleration trajectory sensed by the IMU is at the maximum peak, is 2 in the search area, an intermediate time point between a time point corresponding to the point 1421, which is the highest peak, and a time point corresponding to a point, at which the curve 1420 meets the horizontal axis before the point 1421, may indicate an occurrence time point (or time) of the heel strike event of the right foot, and a time point corresponding to the point 1423, which is the second peak, may indicate an occurrence time point of the toe contact event of the right foot. Since both the heel strike event and the toe contact event have occurred, the electronic device may determine the gait state of the user based on the occurrence time of the heel strike event and the occurrence time of the toe off event.

The graph of FIG. 14 only shows an exemplary change in sensing data according to the gait of the user, and various graphs may appear depending on the gait state of the user and are not limited to the example shown in FIG. 14. For example, when the number of time points, at which the value of the Z-axis acceleration trajectory sensed by the IMU is at the maximum peak, is 1 in the search area, the electronic device may determine that the toe contact event has occurred.

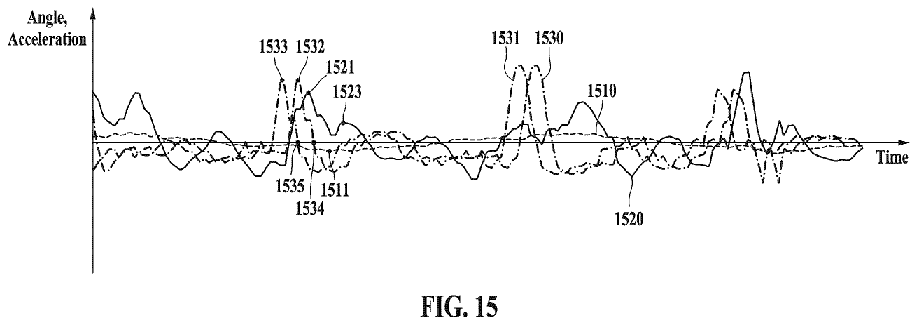

FIG. 15 is a diagram illustrating sensing data for determining a gait state of a user based on a peak time point according to an example.

The graph of FIG. 15 shows a change in sensing data according to the gait of the user. A horizontal axis of the graph indicates time, and a vertical axis indicates a change in hip joint angle or an acceleration measured (or sensed) by an IMU (e.g., the IMU 330 of FIG. 3) of a wearable device (e.g., the wearable device 120 of FIG. 1 or the wearable device 300 of FIG. 3). A curve 1510 may indicate a change in a difference between both hip joint angles, a curve 1520 may indicate a Z-axis acceleration measured by the IMU of the wearable device, a curve 1530 may indicate a first X-axis acceleration measured by the IMU, and a curve 1531 may indicate a second X-axis acceleration measured by the IMU. The curve 1510 may indicate a trajectory of the internal angle change.

Referring to FIG. 15, the electronic device may determine the gait state of the user based on the peak time point corresponding to a point 1511 at which the value of the internal angle change trajectory is at the minimum peak based on the right foot. The electronic device may set the search area having a preset time based on the peak time point corresponding to the point 1511. For example, the search area may be a section that includes the point 1511 and in which the value of the internal angle change trajectory is negative. For example, when the number of time points, at which the value of the Z-axis acceleration trajectory sensed by the IMU is at the maximum peak, is 2 in the search area, an intermediate time point between a point 1521, which is the highest peak, and a time point corresponding to a point, at which the curve 1520 meets the horizontal axis before the point 1521, may indicate an occurrence time point (or time) of the heel strike event of the right foot, and a time point corresponding to the point 1523, which is the second peak, may indicate an occurrence time point of the toe contact event of the right foot. Since both the heel strike event and the toe contact event have occurred, the electronic device may determine the gait state of the user based on the occurrence time of the heel strike event and the occurrence time of the toe off event.

According to an embodiment, referring to the curve 1530, a time point corresponding to a point 1534 at which the value of the first X-axis acceleration trajectory sensed by the IMU passes a point 1532, the maximum peak, and becomes 0 in the search area, may indicate the occurrence time point of the toe off event of the left foot. The electronic device may determine the gait state as the walking state, since the occurrence time of the heel strike event (e.g., corresponding to the point 1521) of the right foot precedes the occurrence time of the toe off event (e.g., corresponding to the point 1534) of the left foot.

According to an embodiment, referring to the curve 1531, a time point corresponding to a point 1535 at which the value of the second X-axis acceleration trajectory sensed by the IMU passes a point 1533, the maximum peak, and becomes 0 in the search area, may indicate the occurrence time point of the toe off event of the left foot. The electronic device may determine the gait state as the first running state, since the occurrence time of the heel strike event (e.g., corresponding to the point 1521) of the right foot is later than the occurrence time of the toe off event (e.g., corresponding to the point 1535) of the left foot.

The graph of FIG. 15 only shows an exemplary change in sensing data according to the gait of the user, and various graphs may appear depending on the gait state of the user and are not limited to the example shown in FIG. 15.

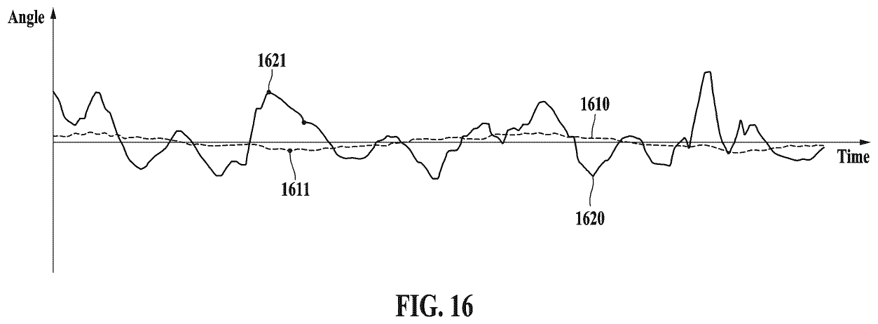

FIG. 16 is a diagram illustrating sensing data for determining a gait state of a user based on a peak time point according to an example.

A graph of FIG. 16 shows a change in sensing data according to the gait of the user. A horizontal axis of the graph indicates time, and a vertical axis indicates a change in hip joint angle or an acceleration measured (or sensed) by an IMU (e.g., the IMU 330 of FIG. 3) of a wearable device (e.g., the wearable device 120 of FIG. 1 or the wearable device 300 of FIG. 3). A curve 1610 may indicate a change in a difference between both hip joint angles, and a curve 1620 may indicate a Z-axis acceleration measured by the IMU of the wearable device. The curve 1610 may indicate a trajectory of the internal angle change.

Referring to FIG. 16, the electronic device may determine the gait state of the user based on the peak time point corresponding to a point 1611 at which the value of the internal angle change trajectory is at the minimum peak based on the right foot. The electronic device may set the search area having a preset time based on the peak time point corresponding to the point 1611. For example, the search area may be a section that includes the point 1611 and in which the value of the internal angle change trajectory is negative. For example, when the number of time points, at which the value of the Z-axis acceleration trajectory sensed by the IMU is at the maximum peak, is 1 in the search area, an intermediate time point between a point 1621, which is the highest peak, and a time point corresponding to a point, at which the curve 1620 meets the horizontal axis before the point 1621, may indicate an occurrence time point (or time) of the toe contact event of the right foot. Since only the toe contact event has occurred, the electronic device may determine the gait state of the user as the second running state. For example, the second running state may refer to a running state that is faster than the first running state. For example, the first running state may indicate a long-distance run (e.g., marathon), and the second running state may indicate a short-distance run.

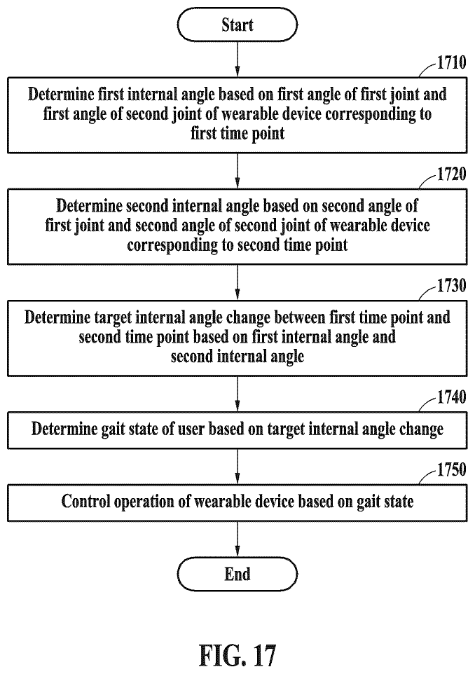

FIG. 17 is a flowchart of a method of controlling an operation of a wearable device according to an embodiment.

According to an embodiment, operations 1710 to 1750 below may be performed by a processor (e.g., the processor 342 of FIG. 3) of a wearable device (e.g., the wearable device 120 of FIG. 1 or the wearable device 300 of FIG. 3).

According to an embodiment, operations 1710 to 1740 below may be performed by a processor (e.g., the processor 220 of FIG. 2) of an electronic device (e.g., the electronic device 110 of FIG. 1 or the electronic device 210 of FIG. 2), and operation 1750 may be performed by the processor of the wearable device. In the above embodiment, the wearable device may obtain (or receive) information about the determined gait state from the electronic device.

In operation 1710, the processor of the electronic device or the wearable device may determine a first internal angle based on a first angle of a first joint and a first angle of a second joint of a wearable device corresponding to a first time point.

In operation 1720, the processor of the electronic device or the wearable device may determine a second internal angle based on a second angle of the first joint and a second angle of the second joint of the wearable device corresponding to a second time point.

In operation 1730, the processor of the electronic device or the wearable device may determine a target internal angle change between the first time point and the second time point based on the first internal angle and the second internal angle.

In operation 1740, the processor of the electronic device or the wearable device may determine a gait state of the user based on the target internal angle change. The method of determining the gait state described with reference to FIG. 8 also applies to operations 1710 to 1740, and a repeated description will be omitted here.

In operation 1750, the processor of the wearable device may control the operation of the wearable device based on the gait state.

According to an embodiment, the processor of the wearable device may control a driver (e.g., the driver 310 of FIG. 3) of the wearable device to provide resistance force to the user. For example, the driver may provide the resistance force to the user by applying an active force to the user via a motor. For example, the driver may provide the resistance force to the user by utilizing the back-drivability of the motor without applying the active force to the user.

According to an embodiment, the processor of the wearable device may control the driver to output a torque (or an assisting torque) for assisting a motion of the user.

According to an embodiment, an electronic device 110; 120; 201; 300 may include a communication module 290; 352 configured to exchange data with an external device. According to an embodiment, the electronic device may include at least one processor 220; 342 configured to control the electronic device. According to an embodiment, the processor may be configured to perform operation 810 of determining a first internal angle based on a first angle of a first joint and a first angle of a second joint of a wearable device corresponding to a first time point. According to an embodiment, the first joint may correspond to an angle of a hip joint of a first leg of a user wearing the wearable device, and the second joint may correspond to an angle of a hip joint of a second leg of the user. According to an embodiment, the processor may be configured to perform operation 820 of determining a second internal angle based on a second angle of the first joint and a second angle of the second joint of the wearable device corresponding to a second time point. According to an embodiment, the processor may be configured to perform operation 830 of determining a target internal angle change between the first time point and the second time point based on the first internal angle and the second internal angle. According to an embodiment, the processor may be configured to perform operation 840 of determining a gait state of the user based on the target internal angle change.

According to an embodiment, the processor may be configured to perform an operation of obtaining the first angle of the first joint and the first angle of the second joint at the first time point from the wearable device. According to an embodiment, the processor may be configured to perform an operation of obtaining the second angle of the first joint and the second angle of the second joint at the second time point from the wearable device.

According to an embodiment, the operation 840 may include operation 1010 of generating an internal angle change trajectory for a gait of the user based on the target internal angle change. According to an embodiment, the operation 840 may include operation 1020 of determining the gait state of the user based on the internal angle change trajectory.

According to an embodiment, the operation 1020 may include operation 1110 of determining whether a predetermined stride event has occurred based on the internal angle change trajectory. According to an embodiment, the operation 1020 may include operation 1120 of increasing the number of strides when it is determined that the stride event has occurred.

According to an embodiment, the predetermined stride event may be an event in which a value of the internal angle change trajectory is changed from a positive number to a negative value at a first time, and the value of the internal angle change trajectory is changed from a positive number to a negative value at a second time that is after the first time.

According to an embodiment, the predetermined stride event may be an event in which a value of the internal angle change trajectory is changed from a negative number to a positive number at a first time, and the value of the internal angle change trajectory is changed from a negative number to a positive number at a second time that is after the first time.

According to an embodiment, the operation 1020 may include operation 1310 of determining the gait state of the user based on a peak time point at which a value of the internal angle change trajectory is at a peak.

According to an embodiment, the operation 1310 may include operation 1312 of setting a search area having a preset time based on the peak time point. According to an embodiment, the operation 1310 may include operation 1314 of determining whether at least one of a heel strike event or a toe contact event has occurred in the search area based on an acceleration value measured by an IMU of the wearable device. According to an embodiment, the operation 1310 may include operation 1318 of determining the gait state of the user based on the at least one of the heel strike event or the toe contact event.

According to an embodiment, the operation 1310 may include operation 1316 of determining whether a toe off event has occurred in the search area based on the acceleration value.

According to an embodiment, the operation 1318 may include an operation of determining the gait state of the user based on an occurrence time of the heel strike event and an occurrence time of the toe off event, when the heel strike event and the toe contact event have occurred.

According to an embodiment, the operation of determining the gait state of the user based on the occurrence time of the heel strike event and the occurrence time of the toe off event, when the heel strike event and the toe contact event have occurred, may include operation 1320 of determining the gait state as a walking state, when the occurrence time of the heel strike event precedes the occurrence time of the toe off event.

According to an embodiment, the operation of determining the gait state of the user based on the occurrence time of the heel strike event and the occurrence time of the toe off event, when the heel strike event and the toe contact event have occurred, may include operation 1330 of determining the gait state as a first running state, when the occurrence time of the heel strike event is later than the occurrence time of the toe off event.

According to an embodiment, the operation 1318 may include operation 1340 of determining the gait state as a second running state, when only the toe contact event has occurred.

According to an embodiment, a method of determining a gait state may include operation 810 of determining a first internal angle based on a first angle of a first joint and a first angle of a second joint of a wearable device 120; 300 corresponding to a first time point. According to an embodiment, the first joint may correspond to an angle of a hip joint of a first leg of a user wearing the wearable device, and the second joint may correspond to an angle of a hip joint of a second leg of the user. According to an embodiment, the method may include operation 820 of determining a second internal angle based on a second angle of the first joint and a second angle of the second joint of the wearable device corresponding to a second time point. According to an embodiment, the method may include operation 830 of determining a target internal angle change between the first time point and the second time point based on the first internal angle and the second internal angle. According to an embodiment, the method may include operation 840 of determining a gait state of the user based on the target internal angle change.

According to an embodiment, the operation 840 may include operation 1010 of generating an internal angle change trajectory for a gait of the user based on the target internal angle change. According to an embodiment, the operation 840 may include operation 1020 of determining the gait state of the user based on the internal angle change trajectory.

According to an embodiment, the operation 1020 may include operation 1110 of determining whether a predetermined stride event has occurred based on the internal angle change trajectory. According to an embodiment, the operation 1020 may include operation 1120 of increasing the number of strides when it is determined that the stride event has occurred.

According to an embodiment, the predetermined stride event may be an event in which a value of the internal angle change trajectory is changed from a positive number to a negative value at a first time, and the value of the internal angle change trajectory is changed from a positive number to a negative value at a second time that is after the first time.

According to an embodiment, the predetermined stride event may be an event in which a value of the internal angle change trajectory is changed from a negative number to a positive number at a first time, and the value of the internal angle change trajectory is changed from a negative number to a positive number at a second time that is after the first time.

According to an embodiment, the operation 1020 may include operation 1310 of determining the gait state of the user based on a time point at which a value of the internal angle change trajectory is at a peak.

According to an embodiment, the operation 1310 may include operation 1312 of setting a search area having a preset time based on the peak time point. According to an embodiment, the operation 1310 may include operation 1314 of determining whether at least one of a heel strike event or a toe contact event has occurred in the search area based on an acceleration value measured by an IMU of the wearable device. According to an embodiment, the operation 1310 may include operation 1318 of determining the gait state of the user based on the at least one of the heel strike event or the toe contact event.

According to an embodiment, a wearable device 120; 300 may include a processor 342 configured to control a wearable device. According to an embodiment, the wearable device may include at least one sensor 321; 321-1 configured to measure an angle of a thigh support frame. According to an embodiment, the wearable device may include a motor driver circuit 312; 312-1 controlled by the processor. According to an embodiment, the wearable device may include a motor 314; 314-1 electrically connected, directly or indirectly, to the motor driver circuit. According to an embodiment, the wearable device may include the thigh support frame configured to transmit a torque generated by the motor to at least a portion of a lower limb of the user. According to an embodiment, the processor may be configured to perform operation 810 of determining a first internal angle based on a first angle of a first joint and a first angle of a second joint of the wearable device corresponding to a first time point. According to an embodiment, the processor may be configured to perform operation 820 of determining a second internal angle based on a second angle of the first joint and a second angle of the second joint corresponding to a second time point. According to an embodiment, the processor may be configured to perform operation 830 of determining a target internal angle change between the first time point and the second time point based on the first internal angle and the second internal angle. According to an embodiment, the processor may be configured to perform operation 840 of determining a gait state of the user based on the target internal angle change. According to an embodiment, the processor may be configured to perform operation 850 of controlling an operation of the wearable device based on the gait state. “Based on” as used herein covers based at least on.

The embodiments described herein may be implemented using a hardware component, a software component and/or a combination thereof. A processing device may be implemented using one or more general-purpose or special-purpose computers, such as, for example, a processor, a controller and an arithmetic logic unit (ALU), a DSP, a microcomputer, a FPGA, a programmable logic unit (PLU), a microprocessor or any other device capable of responding to and executing instructions in a defined manner. The processing device may run an OS and one or more software applications that run on the OS. The processing device also may access, store, manipulate, process, and create data in response to execution of the software. For purpose of simplicity, the description of a processing device is used as singular; however, one skilled in the art will appreciate that a processing device may include multiple processing elements and/or multiple types of processing elements. For example, the processing device may include a plurality of processors, or a single processor and a single controller. In addition, different processing configurations are possible, such as parallel processors.

The software may include a computer program, a piece of code, an instruction, or some combination thereof, to independently or uniformly instruct or configure the processing device to operate as desired. Software and data may be embodied permanently or temporarily in any type of machine, component, physical or virtual equipment, computer storage medium or device, or in a propagated signal wave capable of providing instructions or data to or being interpreted by the processing device. The software also may be distributed over network-coupled computer systems so that the software is stored and executed in a distributed fashion. The software and data may be stored by one or more non-transitory computer-readable recording mediums.

The methods according to the above-described embodiments may be recorded in non-transitory computer-readable media including program instructions to implement various operations of the above-described embodiments. The media may also include, alone or in combination with the program instructions, data files, data structures, and the like. The program instructions recorded on the media may be those specially designed and constructed for the purposes of embodiments, or they may be of the kind well-known and available to those having skill in the computer software arts. Examples of non-transitory computer-readable media include magnetic media such as hard disks, floppy disks, and magnetic tape; optical media such as CD-ROM discs and/or DVDs; magneto-optical media such as optical discs; and hardware devices that are specially configured to store and perform program instructions, such as read-only memory (ROM), random access memory (RAM), flash memory, and the like. Examples of program instructions include both machine code, such as produced by a compiler, and files containing higher-level code that may be executed by the computer using an interpreter.

The above-described devices may be configured to act as one or more software modules in order to perform the operations of the above-described embodiments, or vice versa.

As described above, although the embodiments have been described with reference to the limited drawings, a person skilled in the art may apply various technical modifications and variations based thereon. For example, suitable results may be achieved if the described techniques are performed in a different order, and/or if components in a described system, architecture, device, or circuit are combined in a different manner, or replaced or supplemented by other components or their equivalents. While the disclosure has been illustrated and described with reference to various embodiments, it will be understood that the various embodiments are intended to be illustrative, not limiting. It will further be understood by those skilled in the art that various changes in form and detail may be made without departing from the true spirit and full scope of the disclosure, including the appended claims and their equivalents. It will also be understood that any of the embodiment(s) described herein may be used in conjunction with any other embodiment(s) described herein.

Therefore, other implementations, other embodiments, and equivalents to the claims are also within the scope of the following claims.

1. An electronic device comprising:
a communication module, comprising communication circuitry, configured to exchange data with an external device;
at least one processor, comprising processing circuitry; and
memory comprising one or more storage media storing instructions that, when executed by the at least one processor individually or collectively, cause the electronic device to perform at least:
determining a first internal angle based on a first angle of a first joint and a first angle of a second joint of a wearable device corresponding to a first time point, wherein the first joint corresponds to an angle of a hip joint of a first leg of a user wearing the wearable device, and the second joint corresponds to an angle of a hip joint of a second leg of the user;
determining a second internal angle based on a second angle of the first joint and a second angle of the second joint of the wearable device corresponding to a second time point;
determining a target internal angle change between the first time point and the second time point based on the first internal angle and the second internal angle; and
determining a gait state of the user based on the target internal angle change.

2. The electronic device of claim 1, wherein the instructions, when executed by the at least one processor individually or collectively, further cause the electronic device to perform at least:
obtaining the first angle of the first joint and the first angle of the second joint at the first time point from the wearable device; and
obtaining the second angle of the first joint and the second angle of the second joint at the second time point from the wearable device.

3. The electronic device of claim 1, wherein the determining the gait state of the user based on the target internal angle change comprises:
generating an internal angle change trajectory for a gait of the user based on the target internal angle change; and
determining the gait state of the user based on the internal angle change trajectory.

4. The electronic device of claim 3, wherein the determining the gait state of the user based on the internal angle change trajectory comprises:
determining whether a predetermined stride event has occurred based on the internal angle change trajectory; and
increasing the number of strides when it is determined that the stride event has occurred.

5. The electronic device of claim 4, wherein the predetermined stride event is an event in which a value of the internal angle change trajectory is changed from a positive number to a negative number at a first time, and the value of the internal angle change trajectory is changed from a positive number to a negative number at a second time that is after the first time.

6. The electronic device of claim 4, wherein the predetermined stride event is an event in which a value of the internal angle change trajectory is changed from a negative number to a positive number at a first time, and the value of the internal angle change trajectory is changed from a negative number to a positive number at a second time that is after the first time.

7. The electronic device of claim 3, wherein the determining the gait state of the user based on the internal angle change trajectory comprises:
determining the gait state of the user based on a peak time point at which a value of the internal angle change trajectory is at a peak.

8. The electronic device of claim 7, wherein the determining the gait state of the user based on the peak time point at which the value of the internal angle change trajectory is at the peak comprises:
setting a search area having a preset time based on the peak time point;
determining whether at least one of a heel strike event or a toe contact event has occurred in the search area based on an acceleration value measured by an inertial measurement unit (IMU) of the wearable device; and
determining the gait state of the user based on the at least one of the heel strike event or the toe contact event.

9. The electronic device of claim 8, wherein
the determining the gait state of the user based on the peak time point at which the value of the internal angle change trajectory is at the peak comprises determining whether a toe off event has occurred in the search area based on the acceleration value,
the determining the gait state of the user based on the at least one of the heel strike event or the toe contact event comprises determining the gait state of the user based on an occurrence time of the heel strike event and an occurrence time of the toe off event, when the heel strike event and the toe contact event have occurred.

10. The electronic device of claim 9, wherein the determining the gait state of the user based on the occurrence time of the heel strike event and the occurrence time of the toe off event, when the heel strike event and the toe contact event have occurred, comprises:
determining the gait state as a walking state, when the occurrence time of the heel strike event precedes the occurrence time of the toe off event.

11. The electronic device of claim 9, wherein the determining the gait state of the user based on the occurrence time of the heel strike event and the occurrence time of the toe off event, when the heel strike event and the toe contact event have occurred, comprises:
determining the gait state as a first running state, when the occurrence time of the heel strike event is later than the occurrence time of the toe off event.

12. The electronic device of claim 8, wherein the determining the gait state of the user based on the at least one of the heel strike event or the toe contact event comprises:
determining the gait state as a second running state, when only the toe contact event has occurred.

13. A method comprising:
determining a first internal angle based on a first angle of a first joint and a first angle of a second joint of a wearable device corresponding to a first time point, wherein the first joint corresponds to an angle of a hip joint of a first leg of a user wearing the wearable device, and the second joint corresponds to an angle of a hip joint of a second leg of the user;
determining a second internal angle based on a second angle of the first joint and a second angle of the second joint of the wearable device corresponding to a second time point;
determining a target internal angle change between the first time point and the second time point based on the first internal angle and the second internal angle; and
determining a gait state of the user based on the target internal angle change.

14. The method of claim 13, wherein the determining the gait state of the user based on the target internal angle change comprises:
generating an internal angle change trajectory for a gait of the user based on the target internal angle change; and
determining the gait state of the user based on the internal angle change trajectory.

15. The method of claim 13, wherein the determining the gait state of the user based on the internal angle change trajectory comprises:
determining whether a predetermined stride event has occurred based on the internal angle change trajectory; and
increasing the number of strides when it is determined that the stride event has occurred.

16. The method of claim 15, wherein the predetermined stride event is an event in which a value of the internal angle change trajectory is changed from a positive number to a negative number at a first time, and the value of the internal angle change trajectory is changed from a positive number to a negative number at a second time that is after the first time.

17. The method of claim 15, wherein the predetermined stride event is an event in which a value of the internal angle change trajectory is changed from a negative number to a positive number at a first time, and the value of the internal angle change trajectory is changed from a negative number to a positive number at a second time that is after the first time.

18. The method of claim 14, wherein the determining the gait state of the user based on the internal angle change trajectory comprises:
determining the gait state of the user based on a peak time point at which a value of the internal angle change trajectory is at a peak.

19. The method of claim 18, wherein the determining the gait state of the user based on the peak time point at which the value of the internal angle change trajectory is at the peak comprises:
setting a search area having a preset time based on the peak time point;
determining whether at least one of a heel strike event or a toe contact event has occurred in the search area based on an acceleration value measured by an inertial measurement unit (IMU) of the wearable device; and
determining the gait state of the user based on the at least one of the heel strike event or the toe contact event.

20. A wearable device comprising:
at least one processor, comprising processing circuitry, configured to control a wearable device;
at least one sensor configured to measure an angle of a thigh support frame;
a motor driver circuit configured to be controlled by the at least one processor;
a motor electrically connected to the motor driver circuit;
the thigh support frame configured to transmit a torque generated by the motor to at least a portion of a lower limb of the user; and
memory comprising one or more storage media storing instructions that, when executed by the at least one processor individually or collectively, cause the wearable device to at least:
determining a first internal angle based on a first angle of a first joint and a first angle of a second joint of the wearable device corresponding to a first time point;
determining a second internal angle based on a second angle of the first joint and a second angle of the second joint corresponding to a second time point;
determining a target internal angle change between the first time point and the second time point based on the first internal angle and the second internal angle;
determining a gait state of the user based on the target internal angle change; and
controlling an operation of the wearable device based on the gait state.
